SEQUENCE LISTING (88 sequences)

SEQ ID NO: 1 - DNA, 27 bp, Artificial Sequence
  Synthetic Polynucleotide
gcgcatgcacctttaaaggcgcacgga

SEQ ID NO: 2 - DNA, 30 bp, Artificial Sequence
  Synthetic Polynucleotide
gcctgcagcgttgcatgttggaaccctgga

SEQ ID NO: 3 - DNA, 71 bp, Artificial Sequence
  Synthetic Polynucleotide
gaccttcagcagcttcttcctacatgaccaatactcaatggtggccttctgaattcatggtgagcaagggc

SEQ ID NO: 4 - DNA, 70 bp, Artificial Sequence
  Synthetic Polynucleotide
gagatgtattctgggaaaaattgacatggtatagagtattagagaaatcaaccggcagatcgtcagtcag

SEQ ID NO: 5 - DNA, 69 bp, Artificial Sequence
  Synthetic Polynucleotide
catccacttctggtgatcatcatagcttttattcgcatacagaaacttatagctcaggaggtagcggca

SEQ ID NO: 6 - DNA, 70 bp, Artificial Sequence
  Synthetic Polynucleotide
cacccgattatttgagaaaaacagaaaatatggtacaacttaacagattaaccggcagatcgtcagtcag

SEQ ID NO: 7 - DNA, 25 bp, Artificial Sequence
  Synthetic Polynucleotide
ccacagcaaattcaaacatttcttg

SEQ ID NO: 8 - DNA, 40 bp, Artificial Sequence
  Synthetic Polynucleotide
atggtggcgaccggtgccatacgttcgccgggctgctcgt

SEQ ID NO: 9 - DNA, 40 bp, Artificial Sequence
  Synthetic Polynucleotide
acgagcagcccggcgaacgtatggcaccggtcgccaccat

SEQ ID NO: 10 - DNA, 51 bp, Artificial Sequence
  Synthetic Polynucleotide
gaaagaatataaatatgtaattgtgacatgattatccgcggccgtccttgt

SEQ ID NO: 11 - DNA, 51 bp, Artificial Sequence
  Synthetic Polynucleotide
acaaggacggccgcggataatcatgtcacaattacatatttatattctttc

SEQ ID NO: 12 - DNA, 22 bp, Artificial Sequence
  Synthetic Polynucleotide
gcaggtggtacacataggaaag

SEQ ID NO: 13 - DNA, 24 bp, Artificial Sequence
  Synthetic Polynucleotide
cgagaatgaacaaaaggtttcttc

SEQ ID NO: 14 - DNA, 40 bp, Artificial Sequence
  Synthetic Polynucleotide
atggtggcgaccggtgccatgtcctctgtggagtcacggg

SEQ ID NO: 15 - DNA, 40 bp, Artificial Sequence
  Synthetic Polynucleotide
cccgtgactccacagaggacatggcaccggtcgccaccat

SEQ ID NO: 16 - DNA, 50 bp, Artificial Sequence
  Synthetic Polynucleotide
gtacatttgaaaatttgattcacgatatgcttatccgcggccgtccttgt

SEQ ID NO: 17 - DNA, 50 bp, Artificial Sequence
  Synthetic Polynucleotide
acaaggacggccgcggataagcatatcgtgaatcaaattttcaaatgtac

SEQ ID NO: 18 - DNA, 22 bp, Artificial Sequence
  Synthetic Polynucleotide
tgcagattcggacgaaattggg

SEQ ID NO: 19 - DNA, 22 bp, Artificial Sequence
  Synthetic Polynucleotide
ggaagaagaagcagagaagaaa

SEQ ID NO: 20 - DNA, 49 bp, Artificial Sequence
  Synthetic Polynucleotide
cttcttcaccctttgagaccatgcttcttcaacaaatcataatcgtttg

SEQ ID NO: 21 - DNA, 44 bp, Artificial Sequence
  Synthetic Polynucleotide
gattatgatttgttgaagaagcatggtctcaaagggtgaagaag

SEQ ID NO: 22 - DNA, 46 bp, Artificial Sequence
  Synthetic Polynucleotide
ctcagttggaattctacgaatgctacttatacaattcatccatgcc

SEQ ID NO: 23 - DNA, 46 bp, Artificial Sequence
  Synthetic Polynucleotide
ggcatggatgaattgtataagtagcattcgtagaattccaactgag

SEQ ID NO: 24 - DNA, 24 bp, Artificial Sequence
  Synthetic Polynucleotide
gtctcatgagcggatacatatttg

SEQ ID NO: 25 - DNA, 25 bp, Artificial Sequence
  Synthetic Polynucleotide
ccacagcaaattcaaacatttcttg

SEQ ID NO: 26 - DNA, 40 bp, Artificial Sequence
  Synthetic Polynucleotide
tcatcaatcattaatgtcatacgttcgccgggctgctcgt

SEQ ID NO: 27 - DNA, 48 bp, Artificial Sequence
  Synthetic Polynucleotide
acgagcagcccggcgaacgtatgacattaatgattgatgaaaacttca

SEQ ID NO: 28 - DNA, 50 bp, Artificial Sequence
  Synthetic Polynucleotide
ataaatatgtaattgtgacatgattatcgatgatattgttgttgccattg

SEQ ID NO: 29 - DNA, 48 bp, Artificial Sequence; identical to SEQ ID NO: 27
  Synthetic Polynucleotide

SEQ ID NO: 30 - DNA, 36 bp, Artificial Sequence
  Synthetic Polynucleotide
gattcttgaaaatcccaaagcagcaatttttaccct

SEQ ID NO: 31 - DNA, 36 bp, Artificial Sequence
  Synthetic Polynucleotide
agggtaaaaattgctgctttgggattttcaagaatc

SEQ ID NO: 32 - DNA, 50 bp, Artificial Sequence; identical to SEQ ID NO: 28
  Synthetic Polynucleotide

SEQ ID NO: 33 - DNA, 50 bp, Artificial Sequence
  Synthetic Polynucleotide
acaacaatatcatcgataatcatgtcacaattacatatttatattctttc

SEQ ID NO: 34 - DNA, 25 bp, Artificial Sequence
  Synthetic Polynucleotide
gatgaggagtgccaaaggataaatg

SEQ ID NO: 35 - DNA, 31 bp, Artificial Sequence
  Synthetic Polynucleotide
tcgcaagcttatgatgccacgaatgggacct

SEQ ID NO: 36 - DNA, 31 bp, Artificial Sequence
  Synthetic Polynucleotide
agagaagcttgacgttgttctggcagttggt

SEQ ID NO: 37 - DNA, 21 bp, Artificial Sequence
  Synthetic Polynucleotide
tcgcaagcttcaaagacgctc

SEQ ID NO: 38 - DNA, 32 bp, Artificial Sequence
  Synthetic Polynucleotide
agagaagcttcacattccggaaagctcaattc

SEQ ID NO: 39 - DNA, 31 bp, Artificial Sequence
  Synthetic Polynucleotide
tcgcaagcttatggatccgagtagtccgatg

SEQ ID NO: 40 - DNA, 32 bp, Artificial Sequence
  Synthetic Polynucleotide
agagaagcttcgagatcttctctgatgcttct

SEQ ID NO: 41 - DNA, 32 bp, Artificial Sequence
  Synthetic Polynucleotide
tcgcaagcttgtcctcaacttcagctggaaat

SEQ ID NO: 42 - DNA, 32 bp, Artificial Sequence
  Synthetic Polynucleotide
agagaagcttggagtttccagtccaagatctt

SEQ ID NO: 43 - DNA, 31 bp, Artificial Sequence
  Synthetic Polynucleotide
gcagaagctttggctgcaggagctcaatcat

SEQ ID NO: 44 - DNA, 32 bp, Artificial Sequence
  Synthetic Polynucleotide
agagaagcttcgaatcttcaacgtcattgcca

SEQ ID NO: 45 - DNA, 31 bp, Artificial Sequence
  Synthetic Polynucleotide
gcagaagctttccctaaatcagctgaacagc

SEQ ID NO: 46 - DNA, 31 bp, Artificial Sequence
  Synthetic Polynucleotide
agagaagctttgtgcccatttcaacgaatcc

SEQ ID NO: 47 - DNA, 31 bp, Artificial Sequence
  Synthetic Polynucleotide
tcgcaagcttatgattcgagtgggcacagca

SEQ ID NO: 48 - DNA, 32 bp, Artificial Sequence
  Synthetic Polynucleotide
agagaagcttgcgtaattttgtcgcgattccg

SEQ ID NO: 49 - DNA, 31 bp, Artificial Sequence
  Synthetic Polynucleotide
tcgcaagcttaccttcaactgcagggaatcc

SEQ ID NO: 50 - DNA, 32 bp, Artificial Sequence
  Synthetic Polynucleotide
agagaagcttgaatctccatgtcaaatcaccc

SEQ ID NO: 51 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 52 - protein, 654 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSPDEDEDDLLPPEQKAEREKERRVANNARERLRVRDINEAFKELGRMCQLHLNSEKPQTKLLILHQAVSVILNLEQQVRERNLNPKAACLKRREEEKVSGVVGDPQMVLSAPHPGLSEAHNPAGHM

SEQ ID NO: 53 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 54 - protein, 2174 aa, Homo sapiens
MSASFVPNGASLEDCHCNLFCLADLTGIKWKKYVWQGPTSAPILFPVTEEDPILSSFSRCLKADVLGVWRRDQRPGRRELWIFWWGEDPSFADLIHHDLSEEEDGVWENGLSYECRTLLFKAVHNLLERCLMNRNFVRIGKWFVKPYEKDEKPINKSEHLSCSFTFFLHGDSNVCTSVEINQHQPVYLLSEEHITLAQQSNSPFQVILCPFGLNGTLTGQAFKMSDSATKKLIGEWKQFYPISCCLKEMSEEKQEDMDWEDDSLAAVEVLVAGVRMIYPACFVLVPQSDIPTPSPVGSTHCSSSCLGVHQVPASTRDPAMSSVTLTPPTSPEEVQTVDPQSVQKWVKFSSVSDGFNSDSTSHHGGKIPRKLANHVVDRVWQECNMNRAQNKRKYSASSGGLCEEATAAKVASWDFVEATQRTNCSCLRHKNLKSRNAGQQGQAPSLGQQQQILPKHKTNEKQEKSEKPQKRPLTPFHHRVSVSDDVGMDADSASQRLVISAPDSQVRFSNIRTNDVAKTPQMHGTEMANSPQPPPLSPHPCDVVDEGVTKTPSTPQSQHFYQMPTPDPLVPSKPMEDRIDSLSQSFPPQYQEAVEPTVYVGTAVNLEEDEANIAWKYYKFPKKKDVEFLPPQLPSDKFKDDPVGPFGQESVTSVTELMVQCKKPLKVSDELVQQYQIKNQCLSAIASDAEQEPKIDPYAFVEGDEEFLFPDKKDRQNSEREAGKKHKVEDGTSSVTVLSHEEDAMSLFSPSIKQDAPRPTSHARPPSTSLIYDSDLAVSYTDLDNLFNSDEDELTPGSKKSANGSDDKASCKESKTGNLDPLSCISTADLHKMYPTPPSLEQHIMGFSPMNMNNKEYGSMDTTPGGTVLEGNSSSIGAQFKIEVDEGFCSPKPSEIKDFSYVYKPENCQILVGCSMFAPLKTLPSQYLPPIKLPEECIYRQSWTVGKLELLSSGPSMPFIKEGDGSNMDQEYGTAYTPQTHTSFGMPPSSAPPSNSGAGILPSPSTPRFPTPRTPRTPRTPRGAGGPASAQGSVKYENSDLYSPASTPSTCRPLNSVEPATVPSIPEAHSLYVNLILSESVMNLFKDCNFDSCCICVCNMNIKGADVGVYIPDPTQEAQYRCTCGFSAVMNRKFGNNSGLFLEDELDIIGRNTDCGKEAEKRFEALRATSAEHVNGGLKESEKLSDDLILLLQDQCTNLFSPFGAADQDPFPKSGVISNWVRVEERDCCNDCYLALEHGRQFMDNMSGGKVDEALVKSSCLHPWSKRNDVSMQCSQDILRMLLSLQPVLQDAIQKKRTVRPWGVQGPLTWQQFHKMAGRGSYGTDESPEPLPIPTFLLGYDYDYLVLSPFALPYWERLMLEPYGSQRDIAYVVLCPENEALLNGAKSFFRDLTAIYESCRLGQHRPVSRLLTDGIMRVGSTASKKLSEKLVAEWFSQAADGNNEAFSKLKLYAQVCRYDLGPYLASLPLDSSLLSQPNLVAPTSQSLITPPQMTNTGNANTPSATLASAASSTMTVTSGVAISTSVATANSTLTTASTSSSSSSNLNSGVSSNKLPSFPPFGSMNSNAAGSMSTQANTVQSGQLGGQQTSALQTAGISGESSSLPTQPHPDVSESTMDRDKVGIPTDGDSHAVTYPPAIVVYIIDPFTYENTDESTNSSSVWTLGLLRCFLEMVQTLPPHIKSTVSVQIIPCQYLLQPVKHEDREIYPQHLKSLAFSAFTQCRRPLPTSTNVKTLTGFGPGLAMETALRSPDRPECIRLYAPPFILAPVKDKQTELGETFGEAGQKYNVLFVGYCLSHDQRWILASCTDLYGELLETCIINIDVPNRARRKKSSARKFGLQKLWEWCLGLVQMSSLPWRVVIGRLGRIGHGELKDWSCLLSRRNLQSLSKRLKDMCRMCGISAADSPSILSACLVAMEPQGSFVIMPDSVSTGSVFGRSTTLNMQTSQLNTPQDTSCTHILVFPTSASVQVASATYTTENLDLAFNPNNDGADGMGIFDLLDTGDDLDPDIINILPASPTGSPVHSPGSHYPHGGDAGKGQSTDRLLSTEPHEEVPNILQQPLALGYFVSTAKAGPLPDWFWSACPQAQYQCPLFLKASLHLHVPSVQSDELLHSKHSHPLDSNQTSDVLRFVLEQYNALSWLTCDPATQDRRSCLPIHFVVLNQLYNFIMNML

SEQ ID NO: 55 - protein, 464 aa, Homo sapiens
MDYDFKVKLSSERERVEDLFEYEGCKVGRGTYGHVYKAKRKDGKDDKDYALKQIEGTGISMSACREIALLRELKHPNVISLQKVFLSHADRKVWLLFDYAEHDLWHIIKFHRASKANKKPVQLPRGMVKSLLYQILDGIHYLHANWVLHRDLKPANILVMGEGPERGRVKIADMGFARLFNSPLKPLADLDPVVVTFWYRAPELLLGARHYTKAIDIWAIGCIFAELLTSEPIFHCRQEDIKTSNPYHHDQLDRIFNVMGFPADKDWEDIKKMPEHSTLMKDFRRNTYTNCSLIKYMEKHKVKPDSKAFHLLQKLLTMDPIKRITSEQAMQDPYFLEDPLPTSDVFAGCQIPYPKREFLTEEEPDDKGDKKNQQQQQGNNHTNGTGHPGNQDSSHTQGPPLKKVRVVPPTTTSGGLIMTSDYQRSNPHAAYPNPGPSTSQPQSSMGYSATSQQPPQYSHQTHRY

SEQ ID NO: 56 - protein, 198 aa, Homo sapiens
MAPTCVFLASKVEEFGVVSNTRLIAAATSVLKTRFSYAFPKEFPYRMNHILECEFYLLELMDCCLIVYHPYRPLLQYVQDMGQEDMLLPLAWRIVNDTYRTDLCLLYPPFMIALACLHVACVVQQKDARQWFAELSVDMEKILEIIRVILKLYEQWKNFDERKEMATILSKMPKPKPPPNSEGEQGPNGSQNSSYSQS

SEQ ID NO: 57 - protein, 346 aa, Homo sapiens
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLIF

SEQ ID NO: 58 - protein, 323 aa, Homo sapiens [sequence omitted]

SEQ ID NO: 59 - DNA, 37 bp, Artificial Sequence
  Synthetic Polynucleotide
cgaatttttgcatgcaaaaaacacccactttttgatc

SEQ ID NO: 60 - DNA, 37 bp, Artificial Sequence
  Synthetic Polynucleotide
cgggatcctcgagcacagcatcactttcgtcagcagc

SEQ ID NO: 61 - DNA, 23 bp, Artificial Sequence
  Synthetic Polynucleotide
cgctacagcaaacagcaatttaa

SEQ ID NO: 62 - DNA, 40 bp, Artificial Sequence
  Synthetic Polynucleotide
tggagcctttcctccggtgtctttacgggcggtttgctta

SEQ ID NO: 63 - DNA, 40 bp, Artificial Sequence
  Synthetic Polynucleotide
taagcaaaccgcccgtaaagacaccggaggaaaggctcca

SEQ ID NO: 64 - DNA, 30 bp, Artificial Sequence
  Synthetic Polynucleotide
caatgttttattctctgataaaaagtcaat

SEQ ID NO: 65 - DNA, 23 bp, Artificial Sequence
  Synthetic Polynucleotide
gtgttgttccctttcattttagc

SEQ ID NO: 66 - DNA, 40 bp, Artificial Sequence
  Synthetic Polynucleotide
aggagcttttcctccagtgtctttacgcgcggtttgcttg

SEQ ID NO: 67 - DNA, 40 bp, Artificial Sequence
  Synthetic Polynucleotide
caagcaaaccgcgcgtaaagacactggaggaaaagctcct

SEQ ID NO: 68 - DNA, 23 bp, Artificial Sequence
  Synthetic Polynucleotide
cacacagaaatgcttccaacaaa

SEQ ID NO: 69 - DNA, 40 bp, Artificial Sequence
  Synthetic Polynucleotide
tgtgtcgccgtcgcgtacatacgttcgccgggctgctcgt

SEQ ID NO: 70 - DNA, 46 bp, Artificial Sequence
  Synthetic Polynucleotide
acgagcagcccggcgaacgtatgtacgcgacggcgacacaaaaacg

SEQ ID NO: 71 - DNA, 58 bp, Artificial Sequence
  Synthetic Polynucleotide
gaatataaatatgtaattgtgacatgatcaattaatttcgtcatccgcatcaactggc

SEQ ID NO: 72 - DNA, 52 bp, Artificial Sequence
  Synthetic Polynucleotide
gcggatgacgaaattaattgatcatgtcacaattacatatttatattctttc

SEQ ID NO: 73 - DNA, 40 bp, Artificial Sequence
  Synthetic Polynucleotide
tgtgtgtccgtcgcgtacatacgttcgccgggctgctcgt

SEQ ID NO: 74 - DNA, 46 bp, Artificial Sequence
  Synthetic Polynucleotide
acgagcagcccggcgaacgtatgtacgcgacggacacacaaaaacg

SEQ ID NO: 75 - DNA, 58 bp, Artificial Sequence
  Synthetic Polynucleotide
gaatataaatatgtaattgtgacatgatcaattaatttcgtcatcgtcatcaactggc

SEQ ID NO: 76 - DNA, 52 bp, Artificial Sequence
  Synthetic Polynucleotide
gacgatgacgaaattaattgatcatgtcacaattacatatttatattctttc

SEQ ID NO: 77 - DNA, 40 bp, Artificial Sequence
  Synthetic Polynucleotide
gtatcgtaacgtctactcatacgttcgccgggctgctcgt

SEQ ID NO: 78 - DNA, 44 bp, Artificial Sequence
  Synthetic Polynucleotide
acgagcagcccggcgaacgtatgagtagacgttacgatacaata

SEQ ID NO: 79 - DNA, 43 bp, Artificial Sequence
  Synthetic Polynucleotide
ctcgatcctagtttgatttttgcaatagccacacattcgcccg

SEQ ID NO: 80 - DNA, 47 bp, Artificial Sequence
  Synthetic Polynucleotide
cgggcgaatgtgtggctattgcaaaaatcaaactaggatcgagagaa

SEQ ID NO: 81 - DNA, 49 bp, Artificial Sequence
  Synthetic Polynucleotide
ataaatatgtaattgtgacatgattaatcaaaattcaatcgtcgaacgg

SEQ ID NO: 82 - DNA, 51 bp, Artificial Sequence
  Synthetic Polynucleotide
gacgattgaattttgattaatcatgtcacaattacatatttatattctttc

SEQ ID NO: 83 - DNA, 40 bp, Artificial Sequence; identical to SEQ ID NO: 77
  Synthetic Polynucleotide

SEQ ID NO: 84 - DNA, 44 bp, Artificial Sequence; identical to SEQ ID NO: 78
  Synthetic Polynucleotide

SEQ ID NO: 85 - DNA, 50 bp, Artificial Sequence
  Synthetic Polynucleotide
acctgatgctcgtaatttctgtttggctctccgaagaatcgagccaaacc

SEQ ID NO: 86 - DNA, 52 bp, Artificial Sequence
  Synthetic Polynucleotide
ttcttcggagagccaaacagaaattacgagcatcaggttgtgacaagatggt

SEQ ID NO: 87 - DNA, 49 bp, Artificial Sequence; identical to SEQ ID NO: 81
  Synthetic Polynucleotide

SEQ ID NO: 88 - DNA, 51 bp, Artificial Sequence; identical to SEQ ID NO: 82
  Synthetic Polynucleotide

(19) United States
(12) Patent Application Publication
(10) Pub. No.: US 2020/0123499 A1
(43) Pub. Date: Apr. 23, 2020

(54) METHOD FOR EFFICIENT GENERATION OF NEURONS FROM NON-NEURONAL CELLS

(71) Applicant: Massachusetts Institute of Technology, Cambridge, MA (US)

(72) Inventors: Shuo Luo, Cambridge, MA (US); Howard Robert Horvitz, Wellesley, MA (US)

(73) Assignee: Massachusetts Institute of Technology, Cambridge, MA (US)

(21) Appl. No.: 15/780,765

(22) PCT Filed: Dec. 2, 2016
(86) PCT No.: PCT/US2016/064858

§ 371 Date: Jun. 1, 2018

Related U.S. Application Data
(60) Provisional application No. 62/261,986, filed on Dec. 2, 2015.

(51) Int. Cl. C12N 5/0793

(52) U.S. Cl. CPC C12N 5/0619; C12N 2506/1307; C12N 2501/727; C12N 2501/60

(57) ABSTRACT

This disclosure provides, in part, methods and compositions relating to the genetic reprogramming of non-neuronal cells into neuronal cells. The disclosure further methods and compositions relating to reprogramming neural cells away from the neural fate.

### RELATED APPLICATION

This application claims the benefit under 35 U.S.C. § 119(e) of U.S. Provisional Application Ser. No. 62/261,986 entitled “METHOD FOR EFFICIENT GENERATION OF NEURONS FROM NON-NEURONAL CELLS” filed on Dec. 2, 2015, the entire contents of which are incorporated by reference herein.

### BACKGROUND OF INVENTION

During development, mesoderm and endoderm give rise to primarily non-neural tissues, while neurons are generated mostly from ectoderm. However, some animals such as jellyfish and sea urchins have subsets of neural cells derived from non-ectodermal origins, such as striated muscle and endoderm (1, 2). Neuro-regenerative medicine would benefit from the ability to source neurons from non-neuronal cells.

### SUMMARY OF INVENTION

An understanding of molecular mechanism(s) that generate neurons from non-ectodermal cells would facilitate identification of novel factors useful in neuronal reprogramming. Efficient generation of neurons from non-neuronal cells would also be useful for modeling neurodegenerative diseases and their underlying pathology and developing novel treatments for those diseases. While certain protocols currently exist for neuronal reprogramming, they are unable to achieve reliable and highly efficient neurogenesis.

Provided herein is a novel method to identify genes and gene products (proteins) that promote highly efficient neurogenesis from non-neuronal cells. This method can been used to identify a number of such genes and gene products that together promote highly efficient neural reprogramming. To this end, this disclosure describes an assay to identify genes involved in the reprogramming of the C. elegans mesoderm-derived I4 neuron into a muscle-like cell. This assay, described in greater detail herein, identified transcription factors and a transcriptional coactivator complex that are required for efficient I4 neurogenesis. In particular, the C. elegans homolog of the mammalian ASCL1 proneural protein, HLH3, is expressed in the developing I4 cell and appears to act cell-autonomously to promote I4 neurogenesis. The transcription factor HLH2/TCF3/E2A/Da and the Mediator CDK8 kinase module were found to function synergistically with HLH3 to promote robust I4 neurogenesis. Although not intending to be bound by any particular mechanism or theory, CDK8 may promote I4 neurogenesis by inhibiting the CDK7/CYH1 (CDK7/cyclin H) kinase module of the general transcription initiation factor TFIIH and may also act by phosphorylating Ser10 of the replication independent histone H3.3. These findings reveal a previously unknown role of and mechanism for the Mediator kinase module in promoting non-ectodermal neurogenesis and provide novel candidates in neuroregenerative medicine. As will be described in greater detail herein, these findings identify an important role for proneural proteins and the Mediator CDK8 kinase module in promoting non-ectodermal neurogenesis.

Thus, also provided herein is a novel method for achieving neural reprogramming through the enhanced expression of a subset of genes and proteins in non-neuronal cells. Such genes and gene products include HLH3, CDK8, MED12, MED13 and CIC1. Furthermore, the use of inhibitory agents, particularly inhibitory agents directed to the CDK7/CYH1 complex and its activities, to promote neural cell fate is contemplated. The neuronal cells so generated may be used in further screening assays to identify agents that may work prophylactically or therapeutically to treat a neurodegenerative disease. Alternatively, these neuronal cells may be further differentiated in vitro and may serve as an in vitro model to diagnose and/or study a neurodegenerative disease.

A variety of subsets of genes and gene products may be used and/or targeted to achieve neural reprogramming at high efficiency. Some of these subsets are as follows:

(i) co-expression of ASCL1/HLH3 and CDK8, optionally together with expression of TCF3/HLH2 and/or CYCC/CIC1 and/or other Mediator subunit(s),

(ii) co-expression of ASCL1/HLH3 and CYCC/CIC1, optionally together with expression TCF3/HLH2 and/or of CDK8 and/or other Mediator subunit(s),

(iii) co-expression of ASCL1/HLH3 and TCF3/HLH2, optionally together with expression of CDK8 and/or CYCC/CIC1 and/or other Mediator subunit(s),

(iv) expression of ASCL1/HLH3 and reduced expression of CDK7 and/or CYH-1 (resulting in reduced activity of a CDK7/CYH1 complex), optionally together with expression of TCF3/HLH2 and/or CYCC/CIC1 and/or other Mediator subunit(s);

(v) co-expression of ASCL1/HLH3 and CDK8 protein, and reduced expression of CDK7 and/or CYH-1 (resulting in reduced activity of a CDK7/CYH1 complex), optionally together with expression of TCF3/HLH2 and/or CYCC/CIC1 and/or other Mediator subunit(s); (vi) expression of CDK8 and reduced expression of CDK7 and/or CYH-1 (resulting in reduced activity of a CDK7/CYH1 complex), optionally together with expression of TCF3/HLH2 and/or CYCC/CIC1 and/or other Mediator subunit(s);

(vii) expression of HLH2 and reduced expression of CDK7 and/or CYH-1 (resulting in reduced activity of a CDK7/CYH1 complex), optionally together with expression of CDK8 and/or CYCC/CIC1 and/or other Mediator subunit(s);

(viii) expression of an ASCL1-CDK8 fusion protein, optionally together with expression of TCF3/HLH2 and/or CYCC/CIC1 and/or other Mediator subunit(s),

(ix) expression of an ASCL1-Mediator subunit fusion protein, optionally together with expression TCF3/HLH2 and/or of CDK8 and/or CYCC/CIC1.

Co-expression or expression in the foregoing combinations includes enhanced expression of one or both genes or gene products.

In some instances, the methods involve enhanced expression of one or more endogenous or exogenous CDK8 mediator kinase module proteins such as CDK8, CIC1, MED12 and MED13, or of HLH2.

In some instances, the methods involve reduced expression of endogenous CDK7 and/or CYH1, or reduced activity of CDK7, CYH1 or CDK7/CYH1.

In some instances, the disclosure provides for the combined use of proteins, including those to be upregulated and those to be downregulated in level and ultimately in activity, and that such combined use results in enhanced or synergistic levels of reprogramming. The disclosure contemplates upregulating activity of certain proteins by increasing protein level in a target cell such as a non-neuronal cell. The disclosure further contemplates downregulating activity of other proteins by contacting target cells with inhibitory agents that inhibit the activity of such proteins. Preferably, the inhibitory agents are selective for a particular target such as a particular protein or protein complex.

As will be explained in greater detail herein, the expression may be enhanced expression. For example, a non-neuronal cell may be subject to enhanced expression of one or more or all of the foregoing genes and gene products in order to undergo neural reprogramming. In some instances, the non-neuronal cells may be transduced with an exogenous copy of any one or more or all of the foregoing genes.

Thus, in one aspect, the disclosure provides a method for generating neuronal cells from non-neuronal cells comprising enhancing expression of exogenous or endogenous ASCL1/HLH3 protein and CDK8 protein in non-neuronal cells at a level (or to levels) and for a period of time sufficient for the appearance of neuronal cells. In some embodiments, the method further comprises enhancing expression of exogenous TCF3/HLH2 protein in the non-neuronal cells.

In another aspect, the disclosure provides a method for generating neuronal cells from non-neuronal cells comprising enhancing expression of exogenous or endogenous ASCL1/HLH3 protein, TCF3/HLH2 protein, and CDK8 protein in non-neuronal cells at a level (or to levels) and for a period of time sufficient for the appearance of neuronal cells.

In a further aspect, the disclosure provides a method for generating neuronal cells from non-neuronal cells comprising enhancing expression of exogenous or endogenous ASCL1/HLH3 protein while reducing expression of CDK7 and/or of CYH1, or reducing level of CDK7/CYH1 complex and/or reducing activity of CDK7/CYH-1 complex, in non-neuronal cells at a level (or to levels) and for a period of time sufficient for the appearance of neuronal cells.

In an additional aspect, the disclosure provides a method for generating neuronal cells from non-neuronal cells comprising enhancing expression of exogenous or endogenous ASCL1/HLH3 protein and CDK8 protein while reducing expression of CDK7 and/or of CYH1, or reducing level of CDK7/CYH1 complex and/or reducing activity of CDK7/CYH1 complex, in non-neuronal cells at a level (or to levels) and for a period of time sufficient for the appearance of neuronal cells.

In another aspect, the disclosure provides a method for generating neuronal cells from non-neuronal cells comprising enhancing expression of exogenous or endogenous ASCL1/HLH3 protein and TCF3/HLH2 protein while reducing expression of CDK7 and/or of CYH1, or reducing level of CDK7/CYH complex and/or reducing activity of CDK7/CYH complex, in non-neuronal cells at a level (or to levels) and a period of time sufficient for the appearance of neuronal cells.

In a further aspect, the disclosure provides a method for generating neuronal cells from non-neuronal cells comprising enhancing expression of exogenous or endogenous ASCL1/HLH3 protein, TCF3/HLH2 protein and CDK8 protein while reducing expression of CDK7 and/or of CYH1, or reducing level of CDK7/CYH1 complex and/or reducing activity of CDK7/CYH-1 complex, in non-neuronal cells at a level (or to levels) and a period of time sufficient for the appearance of neuronal cells.

In some embodiments, any of the foregoing methods may further comprise enhancing expression of exogenous or endogenous MED12/DPY22 protein and/or MED13/LET19 protein in the non-neuronal cells.

In some embodiments, any of the foregoing methods may further comprise enhancing expression of exogenous or endogenous CYCC/CIC1 protein in the non-neuronal cells.

In some embodiments, any of the foregoing methods may further comprise reducing expression of endogenous CDK7 and/or CYH1, or reducing level and/or activity of CDK7/CYH1 complex, through the use of one or more CDK7/CYH1 inhibitory agents. CDK7 and/or CYH1 expression levels, such as protein expression levels, may be reduced using RNAi based approaches. CDK7/CYH1 activity may be reduced using CDK7 inhibitors, examples of which are provided herein. Such inhibitors may target the kinase activity of CDK7.

In another aspect, the disclosure provides a method for generating neuronal cells from non-neuronal cells comprising enhancing expression of exogenous ASCL1/HLH3 protein and CYCC/CIC1 protein in non-neuronal cells at a level and for a period of time sufficient for the appearance of neuronal cells. In some embodiments, the method further comprises enhancing expression of exogenous TCF3/HLH2 protein in the non-neuronal cells.

In another aspect, the disclosure provides a method for generating neuronal cells from non-neuronal cells comprising enhancing expression of exogenous ASCL1/HLH3 protein, TCF3/HLH2 protein, and CYCC/CIC1 protein in non-neuronal cells at a level and for a period of time sufficient for the appearance of neuronal cells. In some embodiments, the method further comprises enhancing expression of exogenous MED12/DPY22 protein and/or MED13/LET19 protein in the non-neuronal cells. In some embodiments, the method further comprises enhancing expression of exogenous CDK8 protein in the non-neuronal cells.

In another aspect, the disclosure provides a method for generating neuronal cells from non-neuronal cells comprising enhancing expression of a ASCL1/HLH3-CDK8 fusion protein in non-neuronal cells at a level and a period of time sufficient for the appearance of neuronal cells. In some embodiments, the fusion protein comprises full length ASCL1/HLH3 protein. In some embodiments, the method further comprises enhancing expression of exogenous TCF3/HLH2 protein in the non-neuronal cells. In some embodiments, the method further comprises enhancing expression of exogenous MED12/DPY22 protein and/or MED13/LET19 protein in the non-neuronal cells. In some embodiments, the method further comprises enhancing expression of exogenous CYCC/CIC1 protein in the non-neuronal cells.

In another aspect, the disclosure provides a method for generating neuronal cells from non-neuronal cells comprising enhancing expression of a ASCL1/HLH3-CYCC/CIC1 fusion protein in non-neuronal cells at a level and a period of time sufficient for the appearance of neuronal cells. In some embodiments, the fusion protein comprises full length ASCL1/HLH3 protein. In some embodiments, the method further comprises enhancing expression of exogenous TCF3/HLH2 protein in the non-neuronal cells. In some embodiments, the method further comprises enhancing expression of exogenous MED12/DPY22 protein and/or MED13/LET19 protein in the non-neuronal cells. In some embodiments, the method further comprises enhancing expression of exogenous CDK8 protein in the non-neuronal cells.

In another aspect, the disclosure provides a method for generating neuronal cells from non-neuronal cells comprising enhancing expression of exogenous ASCL1/HLH3 protein and TCF3/HLH2 protein in non-neuronal cells at a level and a period of time sufficient for the appearance of neuronal cells. In some embodiments, the method further comprises enhancing expression of exogenous MED12/DPY22 protein and/or MED13/LET19 protein in the non-neuronal cells. In some embodiments, the method further comprises enhancing expression of exogenous CDK8 protein in the non-neuronal cells. In some embodiments, the method further comprises enhancing expression of exogenous CYCC/CIC1 protein in the non-neuronal cells.

In some embodiments, the non-neuronal cells are fibroblasts. In some embodiments, the non-neuronal cells are hepatocytes. In some embodiments, the non-neuronal cells are muscle lineage cells. In some embodiments, the non-neuronal cells are astrocytes.

In some embodiments, the exogenous proteins or fusion proteins are expressed using a viral expression construct. In some embodiments, the viral expression construct is an adenoviral expression construct or an adenoviral associated expression construct. In some embodiments, the viral expression construct is a CMV expression construct.

In some embodiments, the exogenous proteins are expressed from the same expression construct. In some embodiments, the exogenous proteins are expressed from separate expression constructs.

In some embodiments, the method further comprises enhancing expression of one or more Mediator subunit proteins. In some embodiments, the Mediator subunit protein is selected from the group consisting of MED1, MED4, MED6, MED7, MED8, MED9, MED10, MED11, MED12, MED13, MED13L, MED14, MED15, MED16, MED17, MED18, MED19, MED20, MED21, MED22, MED23, MED24, MED25, MED26, MED27, MED28, MED29, MED30, MED31, CCNC and CDK8.

In some embodiments, the method further comprises enhancing expression of one or more Mediator CDK8 kinase module subunit proteins. In some embodiments, the method further comprises enhancing expression of all Mediator CDK8 kinase module subunit proteins.

In some embodiments, the method further comprises reducing a level of one or more of the foregoing: CDK7 mRNA, CYH1 mRNA, CDK7 protein, CYH1 protein, CDK7 activity, CYH1 activity, CDK7/CYH1 complex, and/or CDK7/CYH1 complex activity. These reductions may be effected through the use of CDK7/CYH1 inhibitory agents and/or RNAi-mediated knockdown approaches, as described herein.

In some embodiments, the neuronal cells are produced with an efficiency of at least 25%.

In some embodiments, the method further comprises differentiating the neuronal cells in vitro. In some embodiments, the method further comprises analyzing the developmental potential of the neuronal cells.

In another aspect, the disclosure provides a method of diagnosing a subject at risk of developing a neurodegenerative disease comprising reprogramming a non-neuronal cell from a subject into a neuronal cell using any of the foregoing methods, including but not limited to by enhancing expression of exogenous or endogenous
(i) ASCL1/HLH3 protein and CDK8 protein;
(ii) ASCL1/HLH3 protein, TCF3/HLH2 protein, and CDK8 protein;
(iii) ASCL1/HLH3 protein and CYCC/CIC1 protein;
(iv) ASCL1/HLH3 protein, TCF3/HLH2 protein, and CYCC/CIC1 protein;
(v) ASCL1/HLH3-CDK8 fusion protein; or
(vi) ASCL1/HLH3-CYCC/CIC1 fusion protein,
   differentiating the neuronal cell in vitro, and analyzing the differentiated neuronal cell for the presence of markers associated with a neurodegenerative disease. In some embodiments, the methods comprise reducing expression and/or activity of endogenous CDK7, CYH1, and/or CDK7/CYH1 complex. Such reductions may be effected through the use of CDK7/CYH1 inhibitory agents and/or RNAi-mediated knockdown approaches.

In some embodiments, the neurodegenerative disease is selected from the group consisting of amyotrophic lateral sclerosis (ALS), Parkinson's disease, Alzheimer's disease, and Huntington's disease. Markers associated with these neurodegenerative diseases include genetic mutations (including those that occur at the genomic level), proteins, protein complexes, and the like. Examples of markers include tau and beta-amyloid proteins in Alzheimer's disease, SOD1 mutations, apolipoprotein E and CNF in ALS, α-synuclein in Parkinson's disease, and PDE10 in Huntington's disease.

In some embodiments, the subject is mammalian. In some embodiments, the subject is human.

These and other aspects and embodiments of the invention will be described in greater detail herein.

### BRIEF DESCRIPTION OF DRAWINGS

FIGS. 1A-1E. The I4 neuron is generated from a C. elegans mesodermal lineage and adopts a pharyngeal muscle cell fate in hlh-3 mutants. (FIG. 1A) Diagram of the MSaa embryonic lineage, which gives rise to the I4 neuron. Neuronal cells are shown in dark grey, and muscle and other mesodermal cells are shown in light grey. The I4 neuron is generated by a mother cell that divides to generate the I4 neuron and the pharyngeal muscle cell pm5. (FIG. 1B) A transcriptional reporter for the C. elegans MyoD gene hlh-1 is expressed in I4 precursors during embryogenesis. (FIG. 1C) Strategy of the genetic screen for mutants in which the presumptive I4 neuron becomes a pm5-like muscle cell. (FIG. 1D) I4 does not express its neuronal identity in hlh-3 mutants. Wild-type I4 exhibited a speckled nuclear morphology characteristic of neurons and expressed the I4-specific neuronal reporter Pnlp-13::gfp as well as the neuronal reporters gfp::rab-3 and Prgef-1::dsRed2 (boxes and insets). By contrast, the I4 cell in an hlh-3(n5469) mutant adopted the fried-egg-like nuclear morphology characteristic of non-neuronal cells and did not express any of the neuronal reporters examined (boxes and insets). (FIG. 1E) The hlh-3 mutant I4 cell adopts the pharyngeal muscle cell fate of its sister pm5. The mutant I4 cell expressed a pm5-specific reporter Pace-1::mCherry as well as pharyngeal muscle reporters Pmyo-2::mCherry::H2B and ceh-22::mCherry, none of which was expressed in wild-type I4 (boxes and insets).

FIGS. 2A-2G. HLH-3 functions synergistically with HLH-2 to promote efficient I4 neurogenesis. (FIG. 2A) Schematic showing HLH-3 protein domains and molecular lesions of the hlh-3 alleles. n5469 and tm1688 are likely null. b, basic domain; HLH, helix-loop-helix. (FIG. 2B) Disruption of HLH-3 results in only partial I4 misspecification. (FIG. 2C) An HLH-3::GFP fusion protein is specifically expressed in wild-type I4 (arrow) but not in its sister pm5 (arrowhead) shortly after their generation. (FIG. 2D) An HLH-2::GFP fusion protein is specifically expressed in wild-type I4 (arrow) but not in its sister pm5 (arrowhead) shortly after their generation. (FIG. 2E) Diagram of the first embryonic cell divisions in wild-type embryos, with I4 and the I4 progenitors circled and the I4-neighbouring progenitors. (FIG. 2F) The neurogenesis of I4 does not require the AB, P2 and E cells, which generate I4 neighbor cells. Laser ablation of AB, P2 and E did not affect I4 GFP expression (arrow), while ablation of EMS (which generates I4) eliminated I4 GFP expression. (FIG. 2G) Introducing an hlh-2 allele into an hlh-3 null mutant (n5469 or tm1688) significantly enhanced I4 misspecification, indicating that HLH-2 functions at least partially in parallel to HLH-3 to promote I4 neurogenesis.

FIGS. 3A-3F. The Mediator CDK8 kinase module consisting of CDK8, CIC1, MED12 and MED13 functions in the same pathway as HLH-2 but in parallel to HLH-3 to promote efficient I4 neurogenesis. (FIG. 3A) The I4 cell in dpy-22 and let-19 mutants adopts a pharyngeal muscle cell fate. The mutant I4 cell showed the fried-egg-like nuclear morphology characteristic of non-neuronal cells (boxes and insets) and expressed the pharyngeal muscle reporter transgene Pmyo-2::mCherry::H2B but not the I4 neuronal reporter transgene Pnlp-13::gfp (boxes and arrows). (FIG. 3B) A GFP reporter transgene driven by the dpy-22 or let-19 promoter is expressed ubiquitously in developing embryos. (FIG. 3C) Schematics showing DPY-22 and LET-19 protein domains and molecular lesions in the dpy-22 and let-19 mutants. (FIG. 3D) The DPY-22 PQ-rich domain interacts with HLH-2. The C-terminal 129 amino acids of the PQ-rich domain deleted in all five dpy-22 alleles were required for the interaction. Δ129, deletion of the last 129 a.a.; c129, the last 129 a.a.; BD, bait vector-only control; AD, prey vector-only control. (FIG. 3E) Introducing an hlh-2 allele into dpy-22 or let-19 mutants does not enhance I4 misspecification. (FIG. 3F) Disruption of dpy-22 or let-19 in an hlh-3 null mutant n5469 significantly enhances I4 misspecification, suggesting that like HLH-2, Mediator functions in parallel to HLH-3 to promote efficient I4 neurogenesis.

FIGS. 4A-4E. CDK-8 promotes I4 neurogenesis partly through H3S10 phosphorylation. (FIG. 4A) Disruption of cdk-8 or cic-1 in an hlh-3 null mutant n5469 enhances I4 misspecification. (FIG. 4B) The kinase activity of CDK-8 is required for promoting I4 neurogenesis. ***, P<0.001. (FIG. 4C) Western blot showing significantly reduced H3S10 phosphorylation in cdk-8; hlh-3 double mutants and rescue by wild-type but not kinase-dead CDK-8 overexpression. (FIG. 4D) Overexpression of a phosphomimetic His3.3 protein HIS-71 but not of His3.1 protein HIS-9 partially suppressed I4 misspecification in cdk-8; hlh-3 double mutants. ***, P<0.001. (FIG. 4E) A model in which an HLH-2/Mediator complex cooperates with the HLH-3 proneural protein to promote I4 neurogenesis at least partly through CDK-8 mediated phosphorylation of H3S10 as well as through inhibition of the CDK7/cyclin H kinase module of TFIIH, a transcription initiation factor. According to this model, the CYH1/CDK7 complex, via its kinase activity, mediates myogenic gene expression and pharyngeal muscle differentiation and thus negatively regulates I4 neurogenesis, and H3S10 phosphorylation partially facilitates neurogenic gene expression in I4.

FIGS. 5A-5B. CDK-8 promotes I4 neurogenesis by inhibiting CYH1 and CDK7. (FIG. 5A) Overexpression of phosphomimetic CYH-1DD but not non-phosphorylatable CYH-IAA using the dpy-22 promoter suppresses I4 defects in cdk-8; hlh-3 mutants. (FIG. 5B) Overexpression of kinase-dead CDK-7KD but not gain-of-function CDK-7EE using the dpy-22 promoter rescues I4 defects in cdk-8; hlh-3 mutants. Mean±s.e.m. *, P<0.05; **, P<0.01; ***, P<0.001 by Student's t-test.

### DETAILED DESCRIPTION OF INVENTION

This disclosure is based, in part, on the findings from a mutational screen that identified a number of genes involved in the specification of the I4 neuron in C. elegans. The significance of the I4 neuron is that it is derived from a non-ectodermal lineage, specifically the muscle lineage. Thus a better understanding of the factors contributing to the neuronal specification of a muscle lineage cell should inform broader attempts to reprogram non-neuronal cells into neuronal cells for research and clinical purposes. It should also inform attempts to prevent neurogenesis or reprogram neural cells away from their neuronal fate.

The assay is described more specifically now. The C. elegans nervous system contains a few neurons that are derived from muscle lineages at a highly efficient rate (100% of time). We developed a novel assay to identify genes that are required to generate one such neuron, known in the art as the I4 neuron (or I4). We used this assay to identify genes and ultimately two co-operating genetic pathways required to generate this neuron. These pathways and the particular genes in each are: (1) ASCL1/HLH3, and (2) TCF3/HLH2, MED12/DPY22, MED13/LET19, CDK8, and CYCC/CIC1. Alteration of pathway 1 and/or pathway 2 may be further supplemented with reduction in the level and/or activity of CDK7/CYH1 complex, in order to further enhance generation of the I4 neuron specifically and neurogenesis more generally.

One of these genes, ASCL1/HLH3, has been implicated in neurogenesis. That this gene was identified in the screening assay described herein serves to validate the assay. Some of the other identified genes were not previously known to play a role in neurogenesis nor in neuronal reprogramming. As a non-limiting example, CDK8 Mediator proteins CDK8, CIC1, MED12 and MED13 have not been previously implicated in neuronal reprogramming. CDK8 has been implicated in cell-fate transformation of several cancers including colon cancer and melanoma. However based on the experimental findings described herein, CDK8 Mediator proteins are candidate targets in a neuronal reprogramming strategy, including such strategies in human cells.

In addition, the interplay of these two transcriptional factor pathways in neurogenesis was also not previously recognized. Enhancing the activity of factors in both pathways should drive neuronal reprogramming in non-neuronal cells. Although either pathway alone is partially sufficient for I4 neurogenesis, the two pathways together promote robust and synergistic I4 neurogenesis from cells of muscle origin.

Accordingly, this disclosure also provides neuronal reprogramming methods that involve enhanced expression of one or more or all of the genes (and gene products) of these pathways. These and other methods further contemplate the use of CDK7/CYH1 inhibitory agents, such as inhibitors that target CDK7, CYH1 and/or CDK7/CYH1 complex, to increase neural reprogramming. These inhibitors include agents that inhibit the kinase activity of CDK7. These methods may also employ RNAi-mediated approaches for reducing protein levels of CDK7, CYH1 and/or the CDK7/CYH1 complex. The ability to reprogram non-neuronal cells facilitates generation of neurons for research and/or clinical uses including diagnostic, prophylactic and/or therapeutic uses.

These various methods are described below in greater detail.

Reprogramming Methods

The disclosure contemplates reprogramming of non-neuronal cells into neuronal cells through expression of one or more reprogramming genes (and their gene products, also referred to herein as proteins). In some instances, the methods provided herein aim to increase the expression and thus level of particular sets of proteins in non-neuronal cells. This may be accomplished through the introduction of nucleotide sequences encoding such proteins into non-neuronal cells. The reprogramming methods described herein may or may not involve direct reprogramming.

Genes and Gene Products

The genes segregate into two pathways, and the reprogramming methods of this disclosure contemplate that at least one gene from each pathway is expressed in order for reprogramming to occur in an efficient manner.

One pathway includes the ASCL1/HLH3 transcription factor. This pathway may be referred to herein as “pathway 1”. ASCL1 is a member of the basic helix-loop-helix (HLH) family of transcription factors. It activates transcription by binding to the E box (5′CANNTG-3′). ASCL1 (Achaete-Scute family bHLH transcription factor) dimerizes with other BHLH proteins in order to bind to DNA efficiently. The 2490 bp mRNA sequence of human ASCL1 can be found at GenBank Accession No. NM_004316. See also Rapa et al. Prostate 73(11): 1241-1249 (2013). The protein sequence is provided as SEQ ID NO: 51.

The other pathway includes the TCF3/HLH2, and CDK8, MED12, MED13 and CYCC/CIC1 proteins (the four of which together form the CDK8 module), as well as other Mediator subunit proteins such as MED1, MED4, MED6, MED7, MED8, MED9, MED10, MED11, MED14, MED5, MED16, MED17, MED18, MED20, MED21, MED22, MED23, MED24, MED25, MED26, MED27, MED28, MED29, MED30, and MED31, among others. This pathway may be referred to herein as “pathway 2”.

TCF3/HLH2 is a member of the E protein (class 1) family of HLH transcription factors. E proteins bind to regulatory E-box sequences as either heterodimers or homodimers, thereby activating transcription from such sequences. Heterodimerization with DNA-binding (class IV) HLH factors can lead to the inhibition of TCF3. Alternatively spliced variants of TCF3 have been reported. The 4451 bp mRNA sequence of transcript variant 1 of human TCF3 can be found at GenBank Accession No. NM_003200. The 4078 bp mRNA sequence of transcript variant 2 of human TCF3 can be found at GenBank Accession No. NM_001136139. See also Goodings et al. Leuk Res 39(1):100-109 (2015). The protein sequence is provided as SEQ ID NO: 52.

Mediator complex (also known as TRAP, SMCC, DRIP or ARC, and referred to herein interchangeably as “Mediator”) is a 1.2 MDa protein aggregate that forms one component of the preinitiation complex. The preinitiation complex is a large protein assembly that partly controls transcriptional initiation through its regulation of most RNA polymerase II (RNAPII) transcripts. It conducts signals from transcription factors to RNAPII, transforming biological inputs from transcription factors into physiological response, as evidenced by changes in gene expression. Mediator binds to a CDK8 subcomplex, also referred to herein interchangeably as a “CDK8 module” or a “Mediator CDK8 kinase module”, which itself comprises CDK8 kinase (also referred to herein interchangeably as “CDK8”), MED12, MED13, and cyclin C. The CDK8 subcomplex modulates Mediator-polymerase II interactions, thereby controlling transcriptional initiation and re-initiation rates.

Mediator complex subunit 12 (MED12), which complexes with CDK8, contributes to the preinitiation complex. MED12 protein activates CDK8 kinase. The 6985 bp mRNA sequence of human MED12 can be found at GenBank Accession No. NM_005120. See also Makinen et al. Int. J Cancer, 134(4):1008-1012 (2014). The protein sequence is provided as SEQ ID NO: 53.

Mediator complex subunit 13 (MED13) is another component of the Mediator complex. MED13 forms a subcomplex with MED12, cyclin C and CDK8. The 10474 bp mRNA sequence of human MED13 can be found at GenBank Accession No. NM_005121. See also Landa et al. PLoS Genet. 5(9): E1000637 (2009). The protein sequence is provided as SEQ ID NO: 54.

CDK8 is a member of the cyclin dependent kinase (CDK) family. CDK8 forms a subcomplex, referred to herein interchangeably as a “CDK8 subcomplex” or a “CDK8 module” or a “Mediator CDK8 kinase module”, with MED12, MED13 and cyclin C. Such subcomplex interacts with and contributes to the Mediator complex. The 1772 bp mRNA sequence of human CDK8 can be found at GenBank Accession No. NM_001260. See also Cooper et al. Dev Cell, 28(2):161-173 (2014). The protein sequence is provided as SEQ ID NO: 55.

Cyclin C is another component of the Mediator complex. Cyclin C forms a subcomplex with MED12, MED13 and CDK8. The 2099 bp mRNA sequence of transcript variant 2 of human cyclin C can be found at GenBank Accession No. NM_001013399. See also Schneider et al., Proc. Natl. Acad. Sci. U.S.A. 110(20), 8081-8086 (2013). The protein sequence is provided as SEQ ID NO: 56.

### Cyclin-Dependent Kinase 7 (CDK7)/Cyclin H (CYH-1) Complex

The methods provided herein, in some instances, involve inhibiting the activity of a complex comprising CDK7 and CYH1 (i.e., referred to herein as a CDK7/CYH1 complex). Such inhibition can be effected through the use of CDK7/CYH1 inhibitory agents. In some embodiments, the methods comprise introducing one or more CDK7/CYH1 inhibitors (or inhibitory agents, as the terms are used interchangeably herein) into the non-neuronal cells (i.e., the target cells). CDK7/CYH1 inhibitors are agents that reduce, in whole or in part, one or more activities of the CDK7/CYH1 complex. Activities of the CDK7/CYH-1 complex include the phosphorylation of the carboxy-terminal domain (CTD) of RNA polymerase II (RNAPII) or the T-loop of cyclin-dependent kinases (CDKs). Inhibition of the activity of CDK7 and/or of the complex can be monitored and/or measured based on the level of CTD phosphorylation and/or T loop phosphorylation, if desired, in some instances. In some instances, the level of CTD phosphorylation is measured as a surrogate for CDK7 inhibition or CDK7/CYH1 inhibition, and this may be particularly suited to measuring CDK7 activity in non-proliferating target cells.

In some instances, the CDK7/CYH1 complex may be inhibited by a CDK7 inhibitor, a cyclin H inhibitor, or both. In some embodiments, the inhibition of CDK7/CYH1 is reversible. In other embodiments, the inhibition of CDK7/CYH1 is irreversible. The CDK7/CYH1 inhibitor may reduce or completely eliminate the ability of the CDK7/CYH1 complex to phosphorylate its substrates, including but not limited to its the carboxy-terminal domain (CTD) of RNA polymerase II (RNAPII). For example, CDK8 inhibits the activity of the CDK7/CYH-1 complex, likely by phosphorylating CYH1. Other agents that phosphorylate and/or inactivate CYH1, and/or bind to and/or inactivate CDK7, including those that prevent, in whole or in part, complex formation in the first place, are contemplated herein. Agents that inhibit, in whole or in part, the kinase activity of CDK7 are also contemplated. Some non-limiting examples of CDK7 inhibitors are presented below.

THZ1, ((E)-N-(3-((5-chloro-4-(1H-indol-3-yl)pyrimidin-2-yl)amino)phenyl)-4-(4-(dimethylamino)but-2-enamido)benzamide), is a selective CDK7 inhibitor. It reportedly modifies CDK7 covalently at Cys312, a residue outside the kinase domain, thereby preventing the phosphorylation of RNAPII CTD (Kwiatkowski et al., Nature, 511(7511):616-20 (2014)). The chemical structure of THZ1 is provided below:

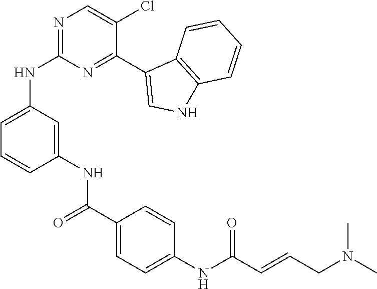 [SMILES: CN(C)C/C=C/C(=O)Nc1ccc(C(=O)Nc2cccc(Nc3ncc(Cl)c(-c4c[nH]c5ccccc45)n3)c2)cc1]

BS-181 is another selective CDK7 inhibitor (Ali et al. Cancer Res, 69(15), 6208-15 (2009)). Its chemical structure is as follows:

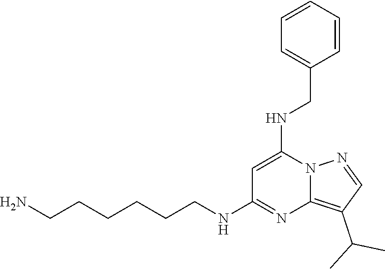 [SMILES: CC(C)c1cnn2c(NCc3ccccc3)cc(NCCCCCCN)nc12]

LDC4297, an (R)—N-(2-(1H-pyrazol-1-yl)benzyl)-8-isopropyl-2-(piperidin-3-yloxy)pyrazolo[1,5-a][1,3,5]triazin-4-amine, is another CDK7 inhibitor. It belongs to the chemical class of pyrazolotriazines (Hutterer et al., Antimicrob. Agents Chemother. 59(4):2062-71 (2015)). Its structure is given below:

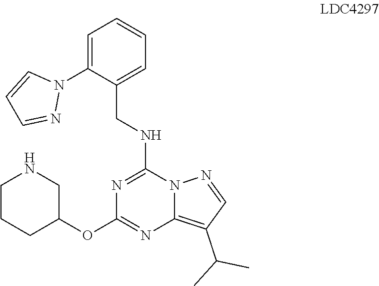 [SMILES: CC(C)c1cnn2c(NCc3ccccc3-n3cccn3)nc(OC3CCCNC3)nc12]

BAY 1000394 ((R)—S-cyclopropyl-S-(4-{[4-{[(1R,2R)-2-hydroxy-1-methylpropyl]oxy}-5-(trifluoromethyl)pyrimidin-2-yl]amino}phenyl)sulfoximide) is yet another CDK7 inhibitor (Lücking et al., Chem Med Chem., 8(7):1067-85 (2013)).

SNS-032 also functions as a CDK7 inhibitor (Cicenas et al., J. Cancer Res. Clin. Oncol. 137(10):1409-18 (2011)). Its chemical structure is given below:

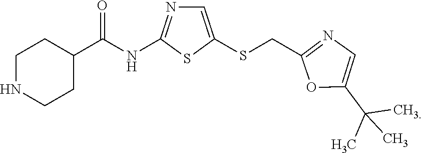 [SMILES: CC(C)(C)c1cnc(CSc2cnc(NC(=O)C3CCNCC3)s2)o1]

VMY-1-101 and VMY-1-103 have also been shown to have CDK7 inhibitory activity (Yenugonda et al., Bioorg Med Chem., 19(8):2714-25 (2011)). Their structures are given below:

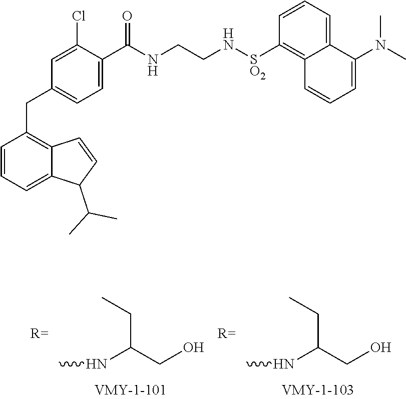

The CDK7 inhibitor may be a compound of the following structure, as fully described in U.S. Pat. No. 9,382,239, the definition of R and other substituents as described therein being incorporated by reference herein:

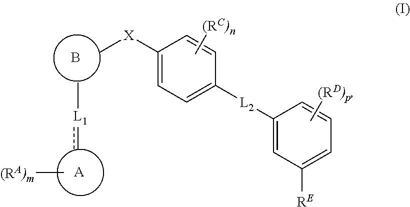

or a pharmaceutically acceptable salt thereof.

The CDK7 inhibitor may be a compound of the following structure, as fully described in U.S. Pat. No. 9,096,608, the definition of R and other substituents as described therein being incorporated by reference herein:

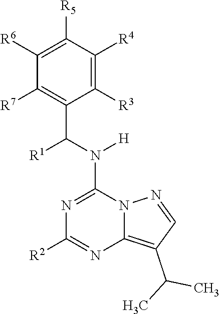

and enantiomers, stereoisomeric forms, mixtures of enantiomers, diastereomers, mixtures of diastereomers, hydrates, solvates, acid salt forms, tautomers, and racemates thereof and pharmaceutically acceptable salts thereof.

Still another CDK7 inhibitor may be a pyrrolopyrimidine carboxamide derivative illustrated below, or a pharmaceutically acceptable salt thereof, as fully described in U.S. Pat. No. 9,062,088, the definition of R and other substituents as described therein being incorporated by reference herein:

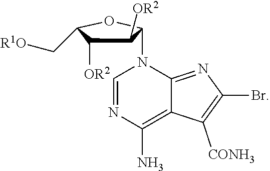

Still another CDK7 inhibitor may be a compound having the following structure, or a pharmaceutically acceptable salt thereof, as fully described in U.S. Pat. No. 6,849,631, the definition of R and other substituents as described therein being incorporated by reference herein:

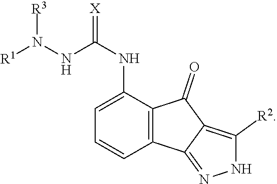

The CDK7 inhibitor may be a compound having the following structure, as fully described in US 2016-0264554, the definition of R and other substituents as described therein being incorporated by reference herein:

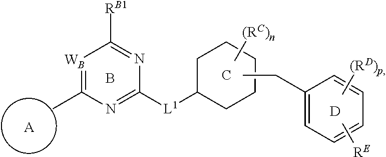

or a pharmaceutically acceptable salt, solvate, hydrate, tautomer, or stereoisomer thereof.

The CDK7 inhibitor may be a compound having the following structure, as fully described in US 2016-0264552, the definition of R and other substituents as described therein being incorporated by reference herein:

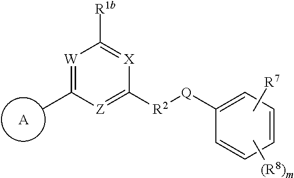

or a pharmaceutically acceptable salt, solvate, hydrate, tautomer, or stereoisomer thereof.

The CDK7 inhibitor may be a compound having the following structure, as fully described in US 2016-0264551, the definition of R and other substituents as described therein being incorporated by reference herein:

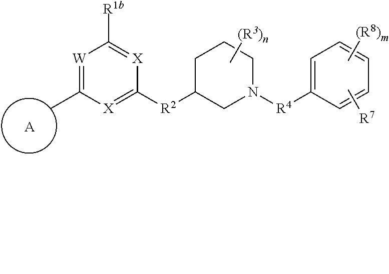

or a pharmaceutically acceptable salt, solvate, hydrate, tautomer, or stereoisomer thereof.

The CDK7 inhibitor may be a compound having the following structure, as fully described in US 2016-0122323, the definition of R and other substituents as described therein being incorporated by reference herein:

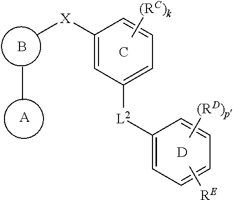

or a pharmaceutically acceptable salt thereof.

The CDK7 inhibitor may be a compound having the following structure, or pharmaceutically acceptable salt, solvate, hydrate, tautomer, or stereoisomer thereof, as fully described in WO 2016/0058544, the definition of R and other substituents as described therein being incorporated by reference herein:

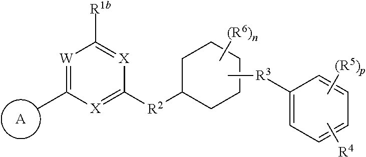

The CDK7 inhibitor may be a compound having the following structure, or an N-oxide thereof, or a pharmaceutically acceptable salt, solvent, polymorph, tautomer, stereoisomer, an isotopic form, or a product of said compound, as fully described in WO 2016/0149031, the definition of R and other substituents as described therein being incorporated by reference herein:

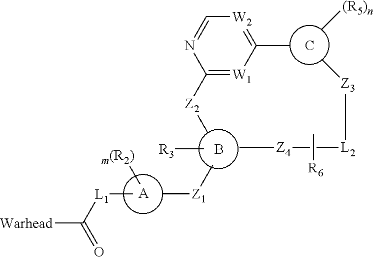

The CDK7 inhibitor may be a compound having the following structure, as fully described in WO 2016/0142855, the definition of R and other substituents as described therein being incorporated by reference herein:

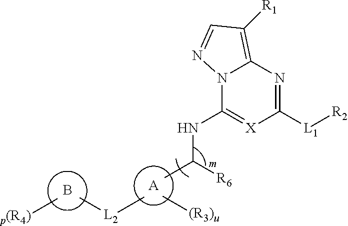

or a pharmaceutically acceptable salt or a stereoisomer thereof;

The CDK7 inhibitor may be any of the following compounds, as described in WO 2016/0058544:

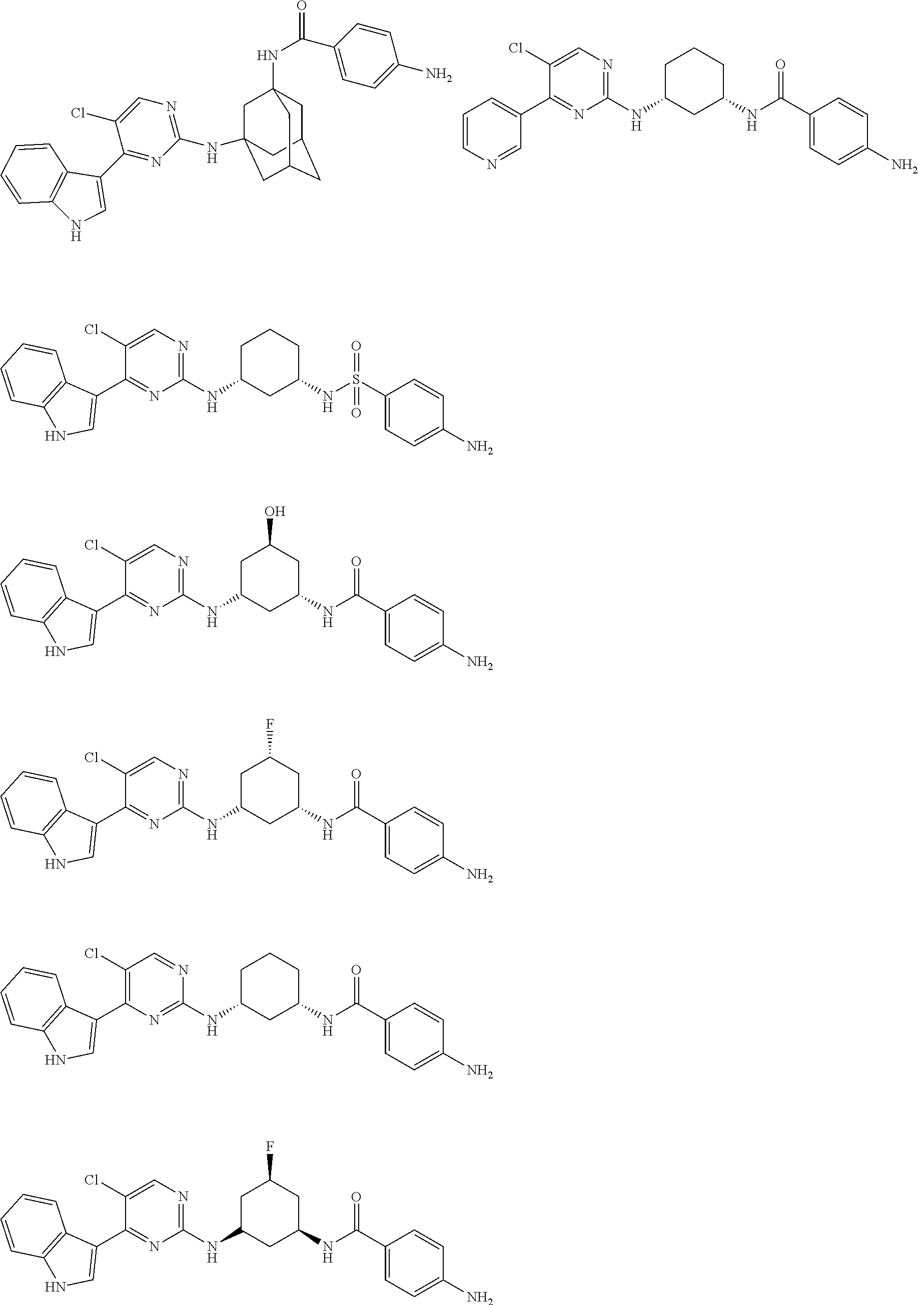

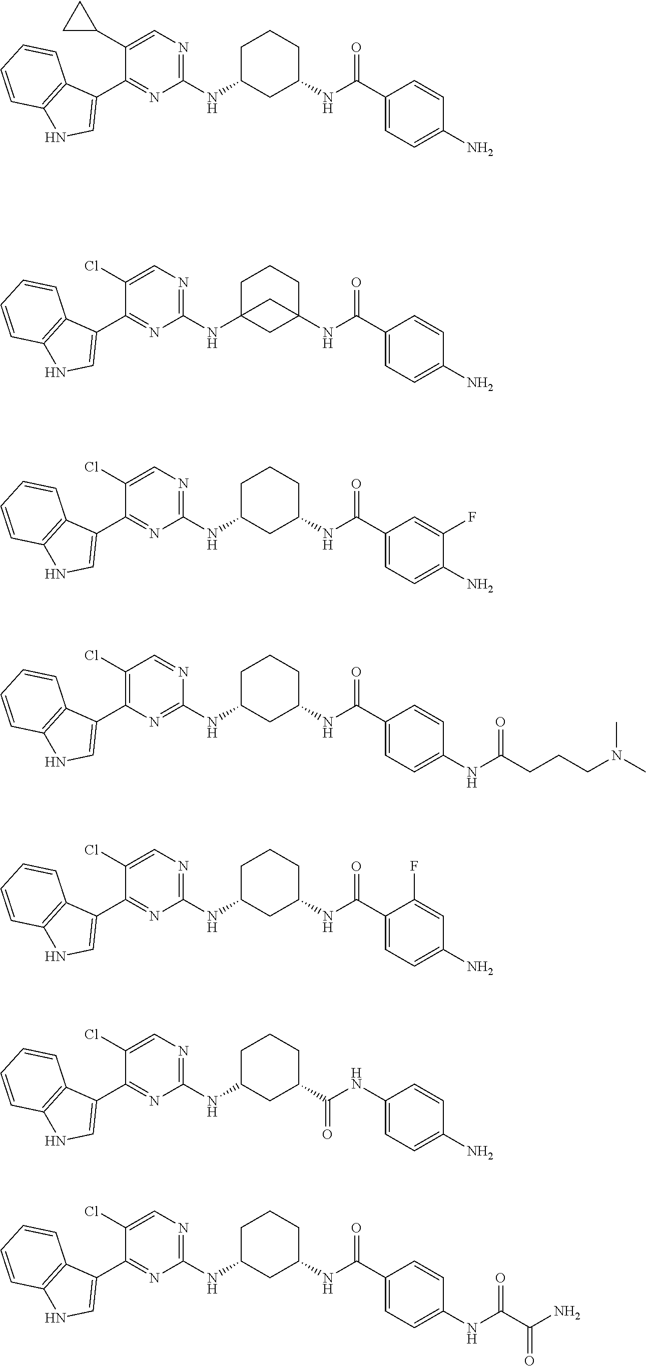

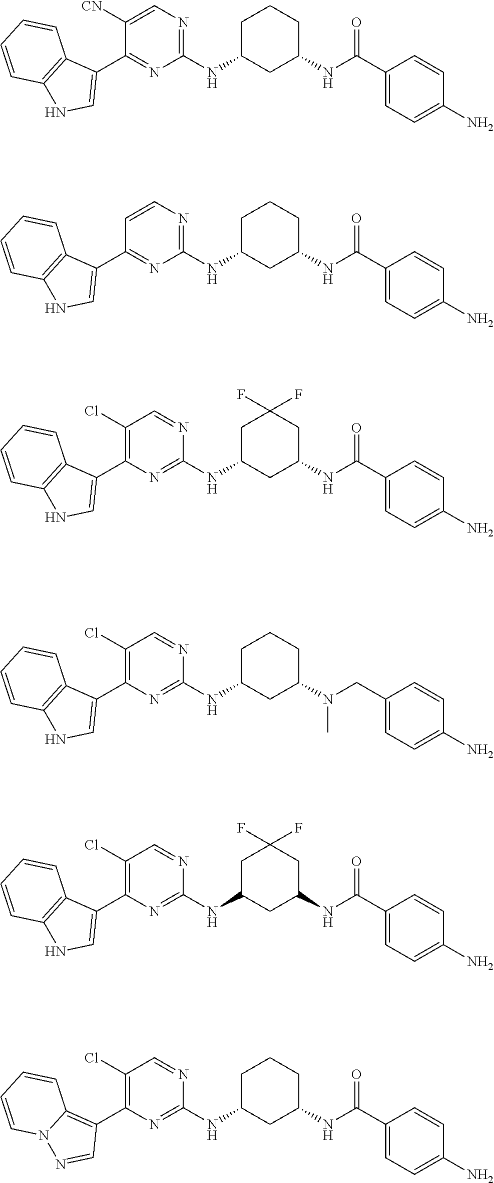

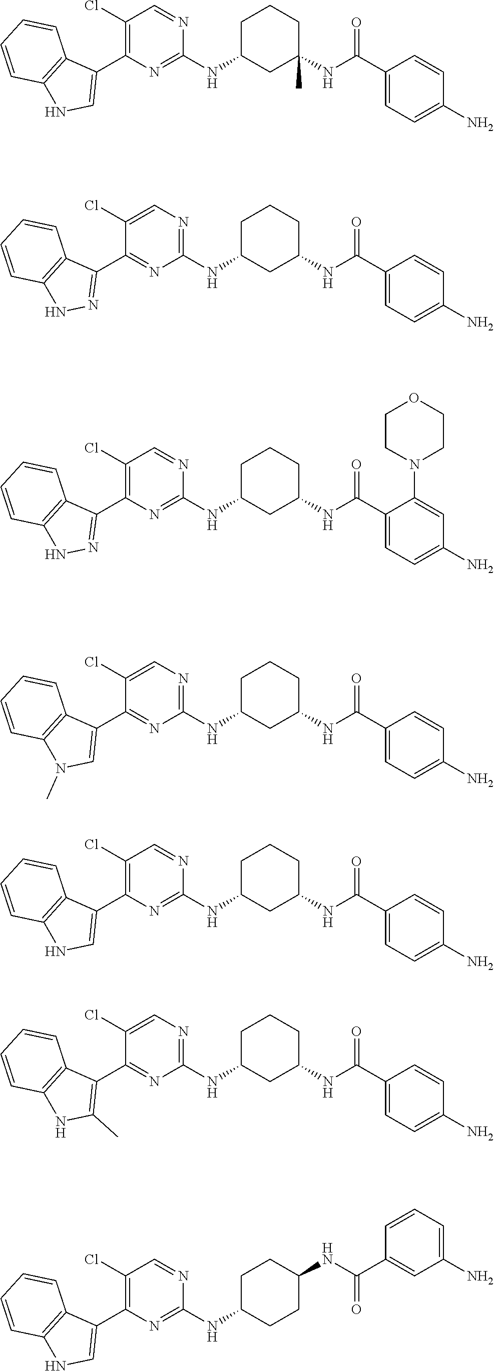

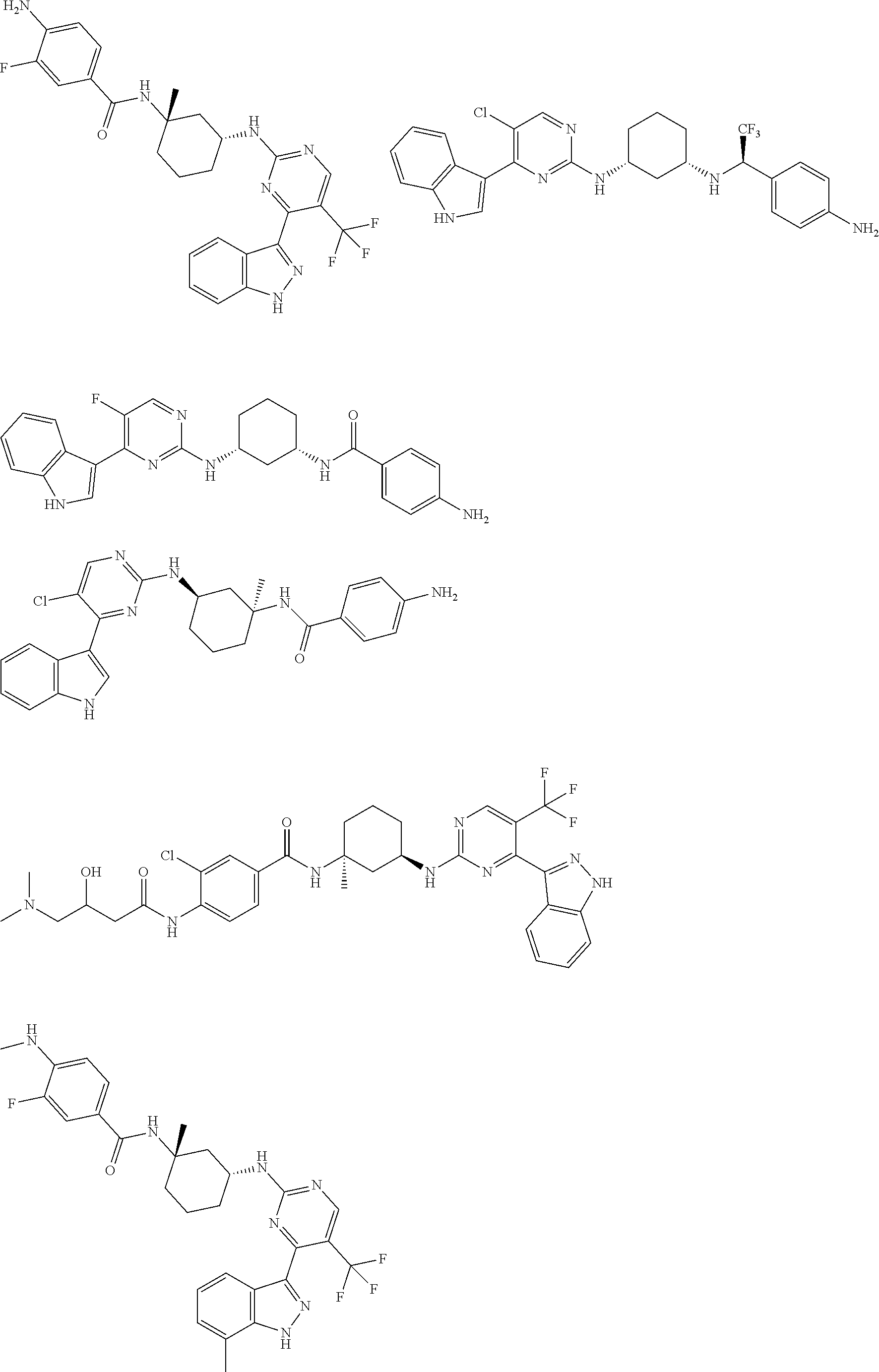 [SMILES: C[C@]1(NC(=O)c2ccc(N)c(F)c2)CCC[C@@H](Nc2ncc(C(F)(F)F)c(-c3n[nH]c4ccccc34)n2)C1 ; Nc1ccc([C@@H](N[C@H]2CCC[C@@H](Nc3ncc(Cl)c(-c4c[nH]c5ccccc45)n3)C2)C(F)(F)F)cc1 ; Nc1ccc(C(=O)N[C@H]2CCC[C@@H](Nc3ncc(F)c(-c4c[nH]c5ccccc45)n3)C2)cc1 ; C[C@]1(NC(=O)c2ccc(N)cc2)CCC[C@@H](Nc2ncc(Cl)c(-c3c[nH]c4ccccc34)n2)C1 ; CN(C)CC(O)CC(=O)Nc1ccc(C(=O)N[C@@]2(C)CCC[C@@H](Nc3ncc(C(F)(F)F)c(-c4n[nH]c5ccccc45)n3)C2)cc1Cl ; CNc1ccc(C(=O)N[C@@]2(C)CCC[C@@H](Nc3ncc(C(F)(F)F)c(-c4n[nH]c5c(C)cccc45)n3)C2)cc1F]

The CDK7 inhibitor may be a compound having the following structure, or a pharmaceutically acceptable salt thereof, as fully described in WO 2016/0105528, the definition of R and other substituents as described therein being incorporated by reference herein:

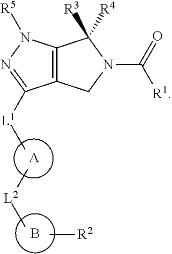

The CDK7 inhibitor may be a compound having the following structure or a pharmaceutically acceptable salt, solvate, hydrate, tautomer, or stereoisomer thereof, as fully described in WO 2015/0154039, the definition of R and other substituents as described therein being incorporated by reference herein:

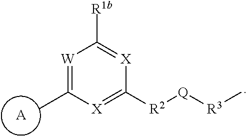

The CDK7 inhibitor may be a compound having the following structure, as fully described in WO 2015/0154022, the definition of R and other substituents as described therein being incorporated by reference herein:

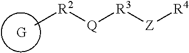

or a pharmaceutically acceptable salt, solvate, hydrate, tautomer, or stereoisomer thereof.

The CDK7 inhibitor may be any one of the following compounds, as described in WO 2015/0154022:

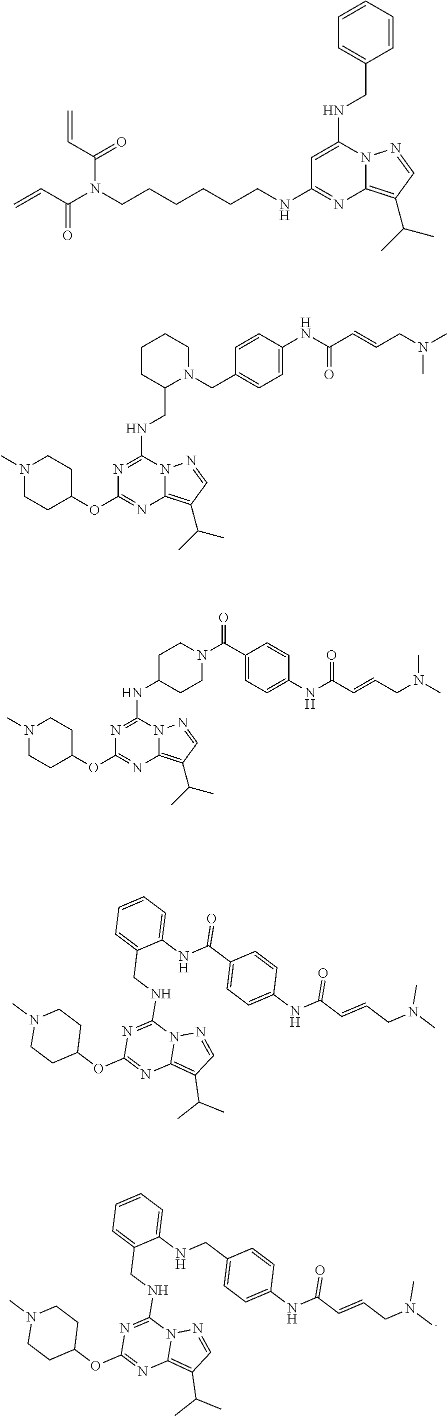

The CDK7 inhibitor may be the following compound, as described in WO 2015/0124941:

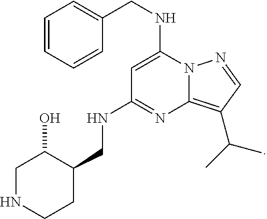 [SMILES: CC(C)c1cnn2c(NCc3ccccc3)cc(NC[C@H]3CCNC[C@@H]3O)nc12]

Other examples of CDK7 inhibitors are discussed in U.S. Pat. Nos. 9,382,239, 9,096,608, 9,062,088, 8,067,424 and 6,849,631; US Patent Application Publication Nos. 2016-0264554, 2016-0264552, 2016-0264551, 2016-0122323 and 2015-0018329; and International Application Publication Nos. WO 2016/0149031, WO 2016/0142855, WO 2016/0058544, WO 2016/0105528, WO 2015/0154039, WO 2015/0154022, and WO 2015/0124941, the specific teachings of which are incorporated by reference herein.

In certain methods provided herein, selective CDK7 inhibitors are used. In other methods, pan CDK inhibitors may be used provided they are able to inhibit CDK7 activity. Examples of such pan CDK inhibitors are provided Kwiatkowski et al., Nature, 511(7511):616-20 (2014), the teachings of which are incorporated herein by reference. Examples include flavopiridol, BMS-387032 (SNS-032), PHA-793887, roscovitine, SCH727965, AZD5438, and AT7519.

As mentioned herein, CDK7 may also be inhibited using RNAi-mediated approaches for reducing CDK7 protein level. The 1,534 bp mRNA sequence of transcript variant 1 of human CDK7 can be found at GenBank Accession No. NM 001799. See also Yang et al., Cell 164(4), 805-17 (2016). The protein sequence is provided as SEQ ID NO: 57.

The present disclosure also contemplates inhibition of cyclin H. An example of a Cyclin H inhibitors include but are not limited to roscovitine and CR8 (S)-isomer (Bettayeb et al., Oncogene 27(44):5797-807 (2008)). As mentioned herein, CYH1 may also be inhibited using RNAi-mediated approaches for reducing CYH1 protein level. The 1,248 bp mRNA sequence of human cyclin H can be found at GenBank Accession No. BC022351. See also Strausberg et al., Proc. Natl. Acad. Sci. U.S.A. 99(26), 16899-16903 (2002). The protein sequence is provided as SEQ ID NO: 58.

CDK7/CYH1 inhibition can be assayed using methods known in the art, including but not limited to radiometric means, immunofluorescence or luminescence, or separation by electrophoresis (gel or microfluidics). Several of these methods are described in Smyth et al., J. Chem. Biol., 2(3): 131-51 (2009). Kinase activity (or lack thereof) can be measured using radiolabeled [³²P]- or [³³P]-ATP, which permits the direct detection of phosphorylation of a substrate peptide or protein by a kinase of interest. The substrate may be the naturally occurring substrate of the CDK7/CYH1 complex or of CYH1, or a suitable fragment thereof. Mobility shift assays can be used to directly measure the phosphorylation of a substrate, and thus the inhibitory ability of an agent, as well. Further, electrophoresis can be used to separate flagged phosphorylated and non-phosphorylated short peptides based on charge, to determine whether phosphorylation of a substrate is inhibited or occurring. The dissociation constant, K_{d}, of an inhibitor-kinase complex can be assayed to determine the affinity of different kinase inhibitor candidates. Methods to determine the K_{d} of a given agent include use of a labeled probe, phage display, and affinity chromatography.

Inhibitor washout experiments may be performed, as described in Kwiatkowski et al., Nature, 511(7511):616-20 (2014). Briefly, cells are incubated with candidate inhibitors for a sufficient period of time (e.g., 4 hours) and temperature (e.g., 37° C.), then washed with saline, and incubated with fresh culture media, without inhibitors, for a second period of time. The cells are then lysed and the resulting lysates are assayed for RNAPII CTD phosphorylation, the absence (or a reduced level) of which is indicative of inhibition.

Inhibition can also be determined by the Lance kinase activity assay, which determines the IC₅₀ values of candidate compounds against CDK/cyclin complexes. The assay is described in US Published Application No. US 2015-0018329, the entire contents of which are incorporated by reference herein. The enzymatic assay uses the phosphorylation of the ULight peptide substrate, which is detected with an anti-phospho-peptide antibody labeled with europium chelate molecules (Eu). When the ULight substrate binds to the Eu antibody, the antibody transfers its dye to the ULight acceptor dye molecule, which emits light at 665 nm. In the presence of kinase inhibitors, phosphorylation of the ULight substrate does not occur, and the signal is diminished or absent.

CDK7/CYH1 inhibitors may be introduced to cells using a variety of techniques known in the art. Target cells may be contacted with inhibitory agents in vitro for sufficient periods of time and under appropriate conditions to facilitate entry of the inhibitory agents into the target cells.

It is to be understood that the reprogramming methods of this disclosure can be performed using the coding sequences specified above or other nucleotide sequences that similarly encode the protein (amino acid) sequences specified above. Thus, the methods may be performed with nucleic acids that encode the proteins of interest and such nucleic acids may be identical to or different from those provided above. Certain variants for example may be variants resulting from the redundancy of the genetic code.

The foregoing sequences are provided for the human homologues to these genes and proteins. However, it is to be understood that the sequences of other mammalian homologues are known and available, and can be used in methods that involve non-human cells as the starting population.

Various embodiments comprise increasing the protein expression and level of

(a) ASCL1/HLH3 by introducing a nucleic acid sequence encoding a ASCL1 protein into a non-neuronal cell, and

(b) TCF3/HLH2 by introducing a nucleic acid sequence encoding a TCF3/HLH2 protein into the non-neuronal cell.

Various embodiments comprise increasing the protein expression and level of

(a) ASCL1/HLH3 by introducing a nucleic acid sequence encoding a ASCL1 protein into a non-neuronal cell, and

(b) TCF3/HLH2 by introducing a nucleic acid sequence encoding a TCF3/HLH2 protein into the non-neuronal cell, and

(c) a Mediator complex subunit protein by introducing a nucleic acid sequence encoding a Mediator complex subunit protein into the non-neuronal cell.

The Mediator complex subunit protein may be CDK8, or it may be MED12, or MED13, or CIC-1. Any combination of any of these may also be used. Alternatively, the Mediator complex subunit protein may be MED1, MED4, MED6, MED7, MED8, MED9, MED10, MED11, MED14, MED15, MED16, MED17, MED18, MED20, MED21, MED22, MED23, MED24, MED25, MED26, MED27, MED28, MED29, MED30, and MED31.

Various embodiments comprise increasing the protein expression and level of

(a) ASCL1/HLH3 by introducing a nucleic acid sequence encoding a ASCL1 protein into a non-neuronal cell, and

(b) CDK8 subcomplex protein by introducing a nucleic acid sequence encoding a CDK8 subcomplex protein into the non-neuronal cell.

The CDK8 subcomplex protein may be CDK8, or it may be MED12, or MED13, or CIC-1. Any combination of any of these may also be used.

Various embodiments comprise increasing the protein expression and level of

(a) ASCL1/HLH3 by introducing a nucleic acid sequence encoding a ASCL1 protein into a non-neuronal cell, and

(b) CDK8 subcomplex protein by introducing a nucleic acid sequence encoding a CDK8 subcomplex protein into the non-neuronal cell, and

(c) TCF3/HLH2 by introducing a nucleic acid sequence encoding a TCF3/HLH2 protein into the non-neuronal cell.

The CDK8 subcomplex protein may be CDK8, or it may be MED12, or MED13, or CIC-1. Any combination of any of these may also be used.

As used herein, the term gene encompasses the coding sequence of a protein of interest. The gene may include intron sequence from the genomic copy of the gene or it may lack such intron sequences. At a minimum, the coding sequence of the protein of interest is to be introduced into the non-neuronal cells. Such coding sequence may be operably linked to a promoter other than that to which it is naturally linked (i.e., its native promoter). Various embodiments provided herein are described in terms of a gene; it is to be understood that the term and such descriptions embrace the use of a coding sequence, optionally without intronic sequences and without native promoter and other transcriptional regulatory sequences. The term gene product typically refers to protein unless otherwise stated.

In some embodiments, the non-neuronal cells are not transduced with a coding sequence for one or more of the following proteins: LHX3, BRN2, MYT1L, ISL1, HB9, NGN2 and NEUROD1. In some embodiments, the non-neuronal cells are not transduced with a coding sequence for one or more of the following proteins: SOX1, PAX6, NKX6.1 and OLIG2.

This disclosure further contemplates methods for promoting neurogenesis through the use of CDK7/CYH1 inhibition alone, or CDK8 mediator kinase module activation alone, or HLH2/TCF3/E2A transcription factor activation alone, as well as any of the foregoing in combination including combinations of any two or of all three.

CDK7/CYH1 inhibition includes reducing CDK7/CYH1 activity which may include reducing expression levels, including protein expression levels, of CDK7 and/or CYH1 or of the CDK7/CYH1 complex. As described herein, CDK7/CYH1 inhibition can be effected by RNAi-mediated knockdown of CDK7 and/or CYH1 protein expression. Alternatively, it can be effected using CDK7 kinase inhibitors such as those described herein and/or known in the art. It can further be effected by using a CDK7 mutant that comprises one or more amino acid changes (additions, deletions, substitutions) that result in reduced kinase activity compared to wildtype CDK7. An example of such a mutant is provided in the Examples.

Reducing, as used herein, includes reducing in whole or in part. Thus, reduction may be complete in which case no expression and/or no activity is detected, or it may be partial. If partial, reduction may be to at least 90%, 80%, 70%, 60%, 50%, 40%, 30%, 20%, 10%, 5%, 1% or less of the level prior to treatment (including of the level in an un-manipulated cell or cell population).

CDK8 mediator kinase module activation includes increasing activity of the CDK8 mediator kinase module which may include increasing expression levels, including protein expression levels, of any one of or any combination of or all of CDK8, CIC1, MED12 and MED13. CDK8 mediator kinase module activity includes but is not limited to phosphorylation of CYH1. The CDK8 kinase phosphorylates CYH1, as explained herein, and thus CDK8 mediator kinase module activation also includes CDK8 kinase activation, intending the activation of the kinase activity of CDK8. CDK8 mediator kinase module activity may be increased, as taught herein, by enhancing (or increasing) expression of the endogenous locus or of an exogenous gene or transcript introduced into the target cell for one or a combination or all of the CDK8 mediator kinase module proteins (i.e., CDK8, CIC1, MED12 and MED13). Additionally, CDK8 kinase activity may be increased by the use of CKD8 mutants that comprise one or more amino acid changes (additions, deletions, substitutions) that result in increased kinase activity compared to wildtype CDK8.

HLH2 activation includes increasing activity of HLH2 which may include increasing expression levels, including protein expression levels, of HLH2. HLH2 activity includes its transcription factor activity and/or its ability to bind to other factors. HLH2 activity may be increased, as taught herein, by enhancing (or increasing) expression of the endogenous HLH2 locus or an exogenous HLH2 gene or transcript introduced into the target cell.

Increasing, as used in the foregoing instances, refers to increasing a transcript or protein level or increasing an activity of the protein or protein complex. The increase is measured relative to the level or activity of the protein or protein complex pre-treatment or the level or activity in an un-manipulated cell or cell population. An increase may be an increase of at least 5%, 10%, 15%, 20%, 25%, 30%, 35%, 40%, 45%, 50%, 55%, 60%, 65%, 70%, 80%, 85%, 90%, 95%, 100%, or more including a 3, 4, 5, 6, 7, 8, 9, 10-fold increase or more relative to pre-treatment levels or level in an un-manipulated cell or cell population.

This disclosure further contemplates methods for preventing or reducing neurogenesis through the use of CDK7/CYH1 activation alone, or CDK8 mediator kinase module inhibition alone, or HLH2/TCF3/E2A transcription factor inhibition alone, as well as any of the foregoing in combination including combinations of any two or of all three.

CDK7/CYH1 activation includes increasing CDK7/CYH1 activity which may include increasing expression levels, including protein expression levels, of CDK7 and/or CYH1 or of the CDK7/CYH1 complex. As described herein, CDK7/CYH1 activation can be effected by enhanced (or increased) expression of the endogenous CDK7 and/or CYH1 loci or of exogenous CDK7 and/or CYH1 genes or transcripts introduced into the target cell. Alternatively, it can be effected using CDK7 kinase activators that act on the CDK7 kinase to enhance its activity. In still other embodiments, it can be effected using CDK7 mutants that comprise one or more amino acid changes (additions, deletions, substitutions) that result in increased kinase activity compared to wildtype CDK7. An example of one such gain-of-function mutant is provided in the Examples.

CDK8 mediator kinase module inhibition includes decreasing activity of the CDK8 mediator kinase module which may include decreasing expression levels, including protein expression levels, of any one of or any combination of or all of CDK8, CIC1, MED12 and MED13. CDK8 mediator kinase module inhibition also includes CDK8 kinase inhibition, intending the inhibition of the kinase activity of CDK8. CDK8 mediator kinase module activity may be decreased, as taught herein, for example using RNAi-mediated approaches to knockdown expression of one or a combination or all of the CDK8 mediator kinase module proteins (i.e., CDK8, CIC1, MED12 and MED13). Additionally, CDK8 kinase activity may be decreased by the use of CKD8 mutants that comprise one or more amino acid changes that result in decreased kinase activity compared to wildtype CDK8. An example of such a mutant is provided in the Examples.

HLH2 inhibition includes decreasing activity of HLH2 which may include decreasing expression levels, including protein expression levels, of HLH2. HLH2 activity may be decreased, as taught herein, by decreasing expression of the endogenous HLH2 locus using or example RNAi-mediated approaches. Other methods for HLH2 inhibition have been described in Snider et al., Mol Cell Bio. 21(5):1866-73 (2001).

CDK8 Inhibitors

Certain CDK8 inhibitors may comprise a truncated cyclin C protein, as fully described in U.S. Pat. No. 6,075,123 and US 2013-0109737, the entire contents of which are incorporated by reference herein. Truncated cyclin C acts as an endogenously encoded cyclin C inhibitor, negatively regulating cyclinC/CDK8 complex activity. Other CKD8 inhibitors include flavopiridol or compound H7 (Rickert et al., Oncogene 18: 1093-1102 (1999).

Another CDK8 inhibitor may be a compound of the following structure, fully described in US 2012-0071477, the definition of R and other substituents as described therein being incorporated by reference herein:

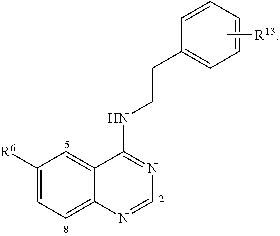

Other CDK8 inhibitors may be compounds having the following structures, as fully described in U.S. Pat. No. 9,321,737, the definition of R and other substituents as described therein being incorporated by reference herein:

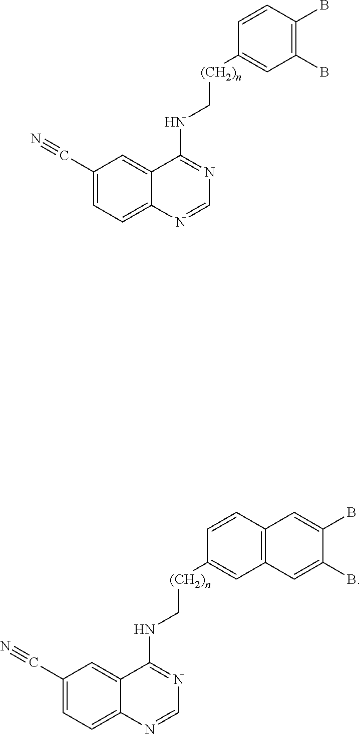

Another CDK8 inhibitor may be a compound having the following structure, as fully described in US 2015-0274726, the definition of R and other substituents as described therein being incorporated by reference herein:

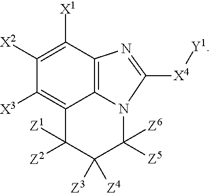

Other CDK8 inhibitors include modified 6-aza-benzothiophene-containing compounds as described in Koehler et al., ACS Med Chem Lett., 7(3): 223-8 (2016), for example:

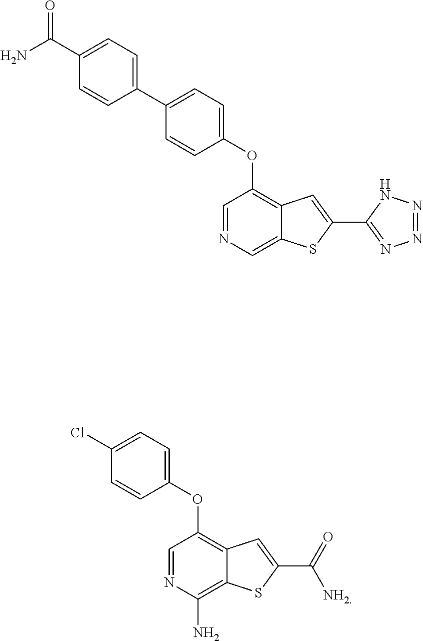 [SMILES: NC(=O)c1ccc(-c2ccc(Oc3cncc4sc(-c5nnn[nH]5)cc34)cc2)cc1 ; NC(=O)c1cc2c(Oc3ccc(Cl)cc3)cnc(N)c2s1]

3-Benzylindazoles can also be modified to be CDK8 inhibitors, as shown below and as described in Schiemann et al., Bioorganic and Medicinal Chem Lett. 26(5):1443-51, the teachings of which are incorporated by reference herein:

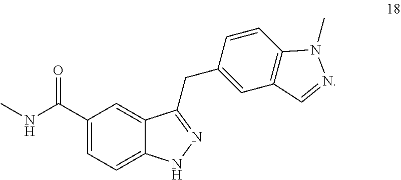 [SMILES: CNC(=O)c1ccc2[nH]nc(Cc3ccc4c(cnn4C)c3)c2c1]

Other CDK8 inhibitors include the following structures, as described in Porter et al., PNAS 109(34):13799-804:

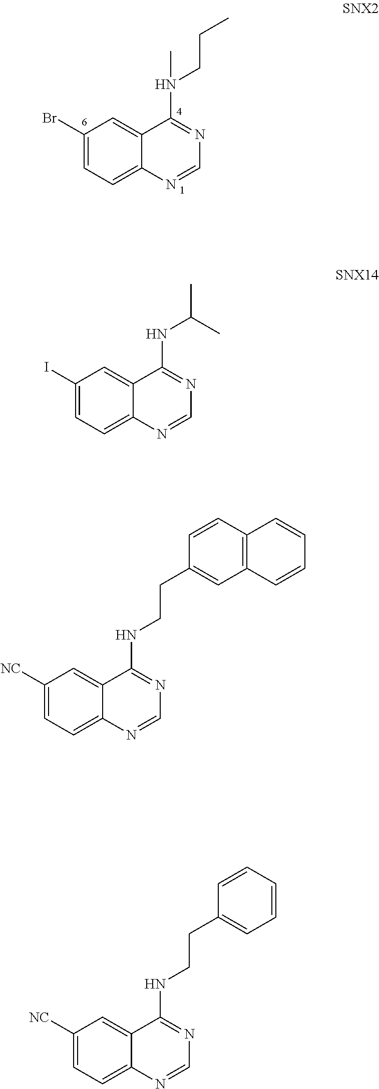 [SMILES: CCCN(C)c1ncnc2ccc(Br)cc12 ; CC(C)Nc1ncnc2ccc(I)cc12 ; N#Cc1ccc2ncnc(NCCc3ccc4ccccc4c3)c2c1 ; N#Cc1ccc2ncnc(NCCc3ccccc3)c2c1]

The CDK8 Mediator kinase complex (or module) may be inhibited by cortistatin A (Pelish et al., Nature 526(7572): 273-6 (2015)).

Other CDK8 inhibitors include those provided in WO 2013/122609, and those inhibitors are incorporated by reference herein.

Enhanced and Increased Expression

In some instances, the methods herein contemplate enhancing expression of at least one pathway 1 and at least one pathway 2 genes in non-neuronal cells. As used herein, enhanced expression includes increasing the expression level of a gene that is already being expressed in the non-neuronal cells. In this case, the enhanced expression level may be about 2, 3, 4, 5, 10, 20, 50, 100 or more times higher than the pre-transduction expression level. In other embodiments, the enhanced expression level is the level of expression of the exogenous gene (or protein) as compared to the level of expression of the endogenous or native gene (or protein). It also includes inducing expression of a gene that is not expressed in the non-neuronal cells. The expression level will typically be assessed on a population basis, and thus will be the average expression level for a population of non-neuronal cells or neuronal cells.

The enhanced expression will typically be effected by introducing the genes of interest into the non-neuronal cells using an expression construct. The genes of interest may be operably linked to inducible or constitutive promoters. Further details regarding various expression constructs and promoters will be provided herein.

In some instances, the methods provided herein contemplate co-expression of one pathway 1 and one or more pathway 2 genes in non-neuronal cells. As used herein, co-expression means that the two or more genes are expressed at overlapping times. The genes may be provided on the same expression construct, optionally under the control of a single promoter or multiple copies of the same promoter. In the former situation, if a single mRNA product is produced that encodes the two or more gene products, then internal ribosome entry sites/sequences (IRES) may be inserted between coding sequences in the expression construct. This helps to ensure a more equivalent level of gene product expression for each gene. Non-viral polycistronic vectors are disclosed in Gonzalez et al., Proc. Natl. Acad. Sci. USA 2009, 106:8918-8922; Carey et al., PNAS, 2009, 106:157-162; WO2009/065618; WO2000/071096; and Okita et al., Science 2008, 322; 949-953.

All of the foregoing methods directed at promoting neurogenesis may further comprise reducing CDK7 and/or CYH1 expression levels and/or activity, including CDK7/CYH1 activity. CDK7 and/or CYH1 activity may be reduced through the inhibition of CDK7, CYH1, and/or the CDK7/CYH1 complex formation. Further methods include increasing the expression (and thus activity) of pathway 1 and/or pathway 2 transcripts and/or gene products while reducing CDK7/CYH1 activity.

Reduced or Decreased Expression

In some instances, the methods disclosed herein refer to reducing expression of particular genes or proteins. Typically, these methods will reduce expression of a particular protein by reducing expression from the endogenous locus that encodes such protein or it may interfere with mRNA transcripts that code for such protein.

One way of reducing expression involves RNA interference (or RNAi). RNAi (also referred to in the art as “gene silencing” and/or “target silencing”, e.g., “target mRNA silencing”) refers to selective intracellular degradation of RNA. RNAi occurs in cells naturally to remove foreign RNA (e.g., viral RNA). Natural RNAi proceeds via fragments cleaved from free dsRNA which direct the degradative mechanism to other similar RNA sequences. This phenomenon can be harnessed and redirected to silence the expression of target genes, for example through the deliberate use of designed nucleic acids.

Double-stranded RNA (dsRNA) when present in a cell are cleaved into ˜20-base pair (bp) duplexes of small-interfering RNAs (siRNAs) by Dicer. siRNAs, either exogenously introduced into a cell or generated by Dicer from shRNA, microRNA, or other substrates bind to the RNA-induced silencing complex (“RISC”), following which they are unwound and the sense strand, also called the “passenger strand” is discarded. The antisense strand of the siRNA, also referred to as the “guide strand”, complexed with RISC then binds to a complementary target sequence, for example, a target sequence comprised in an mRNA, which is subsequently cleaved, resulting in inactivation of the mRNA comprising the target sequence. As a result, the expression of mRNAs containing the target sequence and the corresponding protein expression are reduced.

In vitro and/or in vivo delivery of RNAi reagents are known in the art, and can be used to deliver RNAi constructs. See, for example, U.S. Patent Application Publication Nos. 20160304880, 20160304867, 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654 and 20030157030, and International Application Publication Nos. WO 2008/036825 and WO04/065601.

Proteins that may be downregulated in this manner (or other manners) include pathway 1 proteins such as HLH3, pathway 2 proteins such as HLH2, CDK8, CIC1, MED12, MED13, and the like, as well as CDK7 and CYH1.

RNAi-mediated downregulation of CDK8 is described in WO 2013/122609 and in US Application Publication No. US 2013-0217014, the entire contents of which are incorporated herein by reference.

RNAi-mediated downregulation of HLH3 is described in Thellmann et al., Development 130: 4057-71 (2003), the entire contents of which are incorporated herein by reference.

RNAi-mediated downregulation of HLH2 is described in US Application Publication No. US 2012-0034192, the entire contents of which are incorporated herein by reference.

RNAi-mediated downregulation of CDK7 is described in U.S. Pat. No. 9,012,623, the entire contents of which are incorporated herein by reference.

RNAi-mediated downregulation of CDK7, Cyclin H and MAT1 is described in Patel et al., Clin Cancer Res 22(23):5929-38 (2016), the entire contents of which are incorporated herein by reference.

Suitable shRNA or siRNA for the target of interest can be obtained commercially from a variety of sources including Life Technologies, Open Biosystems, and Ambion.

Exogenous Proteins

In some instances, the methods disclosed herein refer to enhancing expression of exogenous genes or proteins. In this context, exogenous genes or proteins mean those genes that are introduced into the non-neuronal cells via an expression construct and the proteins produced from such introduced genes. The exogenous genes and gene products may be labeled in manner that distinguishes them from their endogenous counterparts. In some instances, the non-neuronal cells do not express the exogenous genes or their gene products, and as a result there is no need to distinguish the native from the exogenous gene expression.

As described in greater detail herein, in some instances, the levels of certain proteins is increased by increasing expression from the endogenous loci that codes for the particular protein.

Proteins that may be upregulated in this manner include pathway 1 proteins, pathway 2 proteins, and in some instances CDK7 and/or CYH1.

Fusion Proteins

The disclosure further contemplates methods involving the expression of fusion proteins comprising proteins from both pathway 1 and pathway 2. The fusion proteins are desirable in some instances since expression of the fusion protein ensures more equivalent expression, of the two or more proteins from pathway 1 and pathway 2. If such fusion proteins comprise either ASCL1/HLH3 or TCF3/HLH2, then it is expected that both proteins will be full length in order to ensure they may still dimerize (either heterodimerize or homodimerize). Typically, the fusion proteins will comprise one but not both of these dimerizing proteins. Even more typically, the fusion protein will comprise ASCL1/HLH3 and not TCF3/HLH2. Thus, examples of fusion proteins include those that comprise full length ASCL1 and CDK8, CYCC/CIC1, MED12 and/or MED13. Thus for example the fusion protein may be a ASCL1-CDK8 fusion protein, or a ASCL-1-CYCC/CIC1 fusion protein, or a ASCL1-MED12 fusion protein, or a ASCL1-MED13 fusion protein. The disclosure thereby provides methods comprising expressing (exogenous) fusion proteins comprising at least one pathway 1 protein and at least one pathway 2 protein. Such methods may further comprise reducing the expression or activity of CDK7 and/or CYH1 and/or the CDK7/CYH1 complex through genetic manipulation and/or the use of inhibitory compounds.

GFP protein has been previously fused to the carboxyl terminal of several bHLH transcription factors like TCF3/HLH-2 and NGN-1 to study their expression pattern (see, for example, Nakano et al., Development. 2010, 137(23):4017-27). A similar strategy may be employed here to form desired fusion proteins (i.e., full length CDK8, CYCC/CIC1, MED12 and/or MED13 may be attached to the C-terminus of ASCL1/HLH-3). Reference may be made to the teachings of Nakano et al. for the details of fusion protein generation, such specific teachings being incorporated by reference herein in their entirety.

Efficiency

The disclosure contemplates methods in which neuronal reprogramming will occur with higher efficiency than is currently possible with available methods. The reported methods achieve at best a reprogramming efficiency of less than 20%, meaning that less than 20% of the non-neuronal cells in the starting population are actually reprogrammed into neuronal cells. The methods provided herein, however, contemplate achieving much higher levels of reprogramming.

Such levels may depend on a number of factors including the nature of the non-neuronal starting cell population, the particular gene combination used, the level of expression or co-expression of such genes, and the like. The efficiency may range from 25% through to 100%, including about 30%, 40%, 50%, 60%, 70%, 80%, 90%, 95%, or 100%.

The methods may be used to reprogram non-neuronal cells in a synergistic manner, intending that the combined use of two or more genes results in a higher efficiency than the additive efficiency obtained when the individual genes are used alone. Such synergy may also be observed through a combined use of a pathway 1 gene or gene product and inhibition of CDK7/CYH1 activity, or a combined use of a pathway 2 gene or gene product and inhibition of CDK7/CYH1 activity, or a combined use of a pathway 1 gene or gene product with a pathway 2 gene or gene product and inhibition of CDK7/CYH1 activity.

Neuronal Cells

The methods may be used to generate one or more types of neuronal cells including motor neurons, sensory neurons, and interneurons. A typical neuron consists of a cell body (referred to as a soma), dendrites, and an axon. The methods may be used to generate cholinergic neurons, GABAergic neurons, glutamatergic neurons, dopaminergic neurons, and/or serotonergic neurons. In particular embodiments, the methods are used to generate motor neurons.

The presence of neuronal cells in the reprogrammed cell population may be determined through the presence of neuronal cell markers. Those markers may vary depending on the species or organism that is used for the starting population. Examples of neuronal cell markers in C. elegans neuronal cells are found in the working examples. Examples of neuronal cell markers in other species or organisms such as humans include transcription factors or structural proteins. Examples of transcription factors include MYT1L, BRN2, SOX1, PAX6, NKX6.1, OLIG2, NGN2, LHX3, ISL1/2, and HB9. Other neuronal markers include tubulin (e.g., Tubb2a and Tubb2b), Map2, Synapsin (e.g., Syn1 and Syn2), synaptophysin, synaptotagmins (e.g., Syt1, Syt4, Syt13, Syt 16), NeuroD, cholineacetyltransferase (ChAT) (e.g., vesicular ChAT), neurofilament, neuromelanin, Tuj1, Thy1, Chat, GluR (kainite 1), Neurod 1, and the like. Expression of receptors for excitatory and inhibitory neurotransmitters can also be used to assess the number and quality of neuronal cells generated.

In addition, gross cell morphology may be used to identify neuronal cells in a population of non-neuronal cells.

The presence of neuronal cells may also be assessed functionally. For example, the cells may be assessed according to electrophysiological characteristics. These assessments may be made using patch-clamp recordings. Other functional characteristics include ability to fire action potentials, produce an outward current in response to glycine, GABA or kainite, and produce an inward current in response to glutamate.

Neuronal cells may be assessed and thus identified by the presence of one or more, including 2, 3, 4, 5, or more, of any of the foregoing characteristics and/or markers.

The neuronal cells or cell population may also be assessed for expression of markers characteristic of the non-neuronal starting cell population. Reprogramming, in some instances, may be evaluated by the increased expression of neuronal markers and decreased expression of markers of the non-neuronal starting cells.

Non-Neuronal Cells

The starting cell population is a non-neuronal cell population. The method envisions that virtually any non-neuronal cell type may be used as the starting cell population. In some instances, it may be desirable to use a starting population that is easily obtainable or accessible. For example, the non-neuronal cells may be fibroblasts such as skin fibroblasts. In other instances, the non-neuronal cell may be a muscle cell.

The non-neuronal starting cell population is typically a somatic cell population. It may be of embryonic or adult origin.

Subjects

The methods may be performed using mammalian cells, including but not limited to human cells. The methods may be performed using non-mammalian cell types and systems such as for example C. elegans.

The subject may be one that has or is at risk of developing a neurodegenerative disease such as a motor neuron disease.

Transduction Methods and Expression Constructs

The non-neuronal cells may be transduced in a variety of ways known in the art. Of particular interest is the use of viral transduction. Examples include adenoviral based transduction and retroviral based transduction.

A nucleic acid vector or construct refers to a nucleic acid into which a nucleic acid sequence of interest can be inserted for introduction into a host cell, such as a non-neuronal cell. Depending on the particular embodiment, such vectors are capable of autonomous replication and/or expression of nucleic acids to which they are linked. An expression vector or construct is a vector or construct that is capable of directing the expression of coding sequences carried in the vector. An expression vector comprises the necessary regulatory regions needed for expression of a coding sequence of interest in a host cell. In some embodiments the coding sequence of interest is operably linked to another sequence in the vector. Vectors can be viral vectors or non-viral vectors. Viral vectors may be replication defective, in which case they lack all viral nucleic acids required for replication. A replication defective viral vector will still retain its infective properties and ability to enter host cells in a similar manner as a replication competent vector, however once in the cell a replication defective viral vector does not reproduce or multiply.

Vectors also encompass liposomes and nanoparticles and other means to deliver DNA molecule to a cell.

The term “operably linked” means that the regulatory sequences necessary for expression of the coding sequence are in the appropriate positions relative to the coding sequence so as to effect expression of the coding sequence. This same definition may apply to the arrangement of coding sequences and transcription control elements (e.g. promoters, enhancers, and termination elements) in an expression vector. The term may include having an appropriate start signal (e.g., ATG) at the beginning of the coding sequence to be expressed, and maintaining the correct reading frame to permit expression of the entire coding sequence.

Viral vectors refer to viruses or virus-associated vectors used to introduce a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for transduction into cells. The vector may or may not be incorporated into the cell's genome. The constructs may include viral sequences for transfection, if desired. Alternatively, the construct may be incorporated into vectors capable of episomal replication, e.g. EPV and EBV vectors.

Retroviral vectors incorporate into the host cell genome and can potentially disrupt normal gene function. In contrast, non-integrating vectors control expression of a gene product by extrachromosomal transcription. Non-integrating vectors do not become part of the host genome, and therefore they tend to express a nucleic acid transiently in a cell population, due in part to the fact they are typically replication deficient. Non-integrating vectors have several advantages over retroviral vectors including but not limited to: (1) no disruption of the host genome, and (2) transient expression, and (3) no remaining viral integration products. Examples of non-integrating vectors include adenovirus, baculovirus, alphavirus, picomavirus, and vaccinia virus. In one embodiment, the non-integrating viral vector is an adenovirus. Non-integrating viral vectors offer further advantages such as their ability to be produced in high titers, their stability in vivo, and their efficient infection of host cells.

Regulatory sequences are nucleic acid sequences, such as initiation signals, enhancers, and promoters, which induce or control transcription of coding sequences to which they are operatively linked. The coding sequences introduced into a non-neuronal cell may be under the control of regulatory sequences which are the same or which are different from those regulatory sequences which control transcription of the naturally-occurring form of a protein. Preferably, the promoter sequence is recognized by the synthetic machinery of the cell, or introduced synthetic machinery, required for initiating transcription of a specific gene.

Diagnostic and Research Methods

The neuronal cells generated using the transduction methods provided herein may be used to study progression of neurodegenerative diseases. The cells may be used in a screening assay to identify agents that may contribute or cause neurodegenerative disease. If the cells are derived from a human subject, they may be used to assess if the subject is at risk of neurodegenerative disease by allowing the cells to differentiate in vitro with or without candidate neurodegenerative triggers and analyzing their developmental potential and/or disease progression.

Neurodegenerative Diseases

The methods can be used in the diagnosis or study of neurodegenerative diseases. Examples of neurodegenerative diseases include but are not limited to Parkinson's disease Alzheimer's disease, Spinal muscular atrophy (SMA), including Type I (also called Werdnig-Hoffmann disease), Type II, Type III (Kugelberg-Welander disease), amyotrophic lateral sclerosis (ALS), Charcot-Marie-Tooth disease (CMT), Progressive bulbar palsy, Pseudobulbar palsy, Primary lateral sclerosis (PLS), Progressive muscular atrophy, Fazio-Londe disease, Kennedy's disease also known as progressive spinobulbar muscular atrophy; congenital SMA with arthrogryposis, Post-polio syndrome (PPS) and traumatic spinal cord injury. In some embodiments, the disease is a motor neuron disease such as SMA and ALS.

Compositions and Kits

This disclosure further contemplates and provides compositions comprising the neuronal cells produced according to the methods provided herein. Such compositions may be pharmaceutical compositions that are suitable for used in vivo.

Additional compositions include kits that comprise coding sequences for any of the foregoing subsets of genes, optionally provided in vectors such as expression vectors. Each coding sequence may be provided in a separate expression vector or two or more coding sequences may be provided in a single expression vector.

In Vivo Uses

The disclosure further contemplates transduction of non-neuronal cells into neuronal cells in vivo using gene therapy approaches. Also contemplated is the use of in vitro generated neuronal cells in an in vivo setting such as for prophylactic or therapeutic purpose.

The following Examples are included for purposes of illustration and are not intended to limit the scope of the invention.

### EXAMPLES

### Materials and Methods

### Strains.

All C. elegans strains were handled and maintained at 22° C. as described previously (56) unless noted otherwise. We used the Bristol strain N2 as the wild-type strain. The mutations used are listed below:
LGI: cdk-8(tm1238), hlh-2(bx115, n5287, tm768ts).
LGII: hlh-3(n5469, n5564, n5566, ot354, tm1688), let-19(n5470, n5563, ok331), oxIs322[Pmyo-2::mCherry:: H2B, Pmyo-3::mCherry:: H2B, Cbr-unc-119(+)].
LGIII: cic-1(tm3740), cnd-1(gk718, gk781), jsIs682[gfp::rab-3, lin-15(+)], otIs173[Prgef-1::dsRed2, Pttx-3::gfp], nIs695[ceh-22::mCherry, Ppgp-12::mCherry].
LGIV: ngn-1(ok2200), nIs198[Punc-25::mStrawberry, lin-15(+)], nIs407[hlh-2::gfp, lin-15(+)].
LGV: nIs310[Pnlp-13:.gfp, lin-15(+)], nIs662[hlh-3::gfp, Punc-54::mCherry], otIs292[eat-4::mCherry, rol-6(su1006)].
LGX: dpy-22(bx92, e652, n5571, n5572, n5573, n5574, n5662, sy622), nIs116[Pcat-2::gfp, lin-15(+)], vsIs48[Punc-17: gfp].
unknown linkage: nIs324[Ptdc-1::mStrawberry, lin-15(+)], nIs625[Pdpy-22::gfp], nIs626[Plet-19::gfp].
Extrachromosomal arrays: nEx2343[Pace-1::mCherry], nEx2227[Pdpy-22::cdk-8(cDNA)::dpy-22 3′-UTR, Punc-54::mCherry], nEx2228[Pdpy-22::cdk-8(cDNA, KD)::dpy-22 3′-UTR, Punc-54::mCherry].

### Molecular Biology and Fluorescence Reporters.

The Pmyo-2::mCherry:: H2B, Prgef-1::dsRed2, Punc-17::gfp transcriptional reporters and the eat-4::mCherry, gfp::rab-3, hlh-2::gfp translational reporters have been described previously (4, 5, 7, 25)'(57, 58). The Pnlp-13:: gfp transcriptional reporter was constructed by PCR amplifying a 2.3 kb nlp-13 promoter fragment with the oligonucleotides

fw-GCGCATGcacctttaaaggcgcacgga (SEQ ID NO: 1) and
rv-GCCTGCAGCGTTGCATgttggaaccctgga (SEQ ID NO: 2).

The resulting product was digested by SphI and PstI and cloned into pPD95.75 digested by the same restriction enzymes. The plasmid was subsequently injected into the germ line of wild-type animals to generate transgenic strains.

The Punc-25::mStrawberry transcriptional reporter was made by PCR-amplification using the two primers

fw-cgaatttttgcatgcaaaaaacacccactttttgatc (SEQ ID NO: 59) and rv-CGGGATCCTCgagcacagcatcactttcgtcagcagc (SEQ ID NO: 60). The resulting PCR product was digested by SphI and BamHI and cloned into pSN199 digested by the same enzymes. pSN199 is a derivative of pPD122.56 carrying mStrawberry instead of GFP. In short, pSN199 was made by replacing GFP of pPD122.56 with mStrawberry from the plasmid mStrawberry 6. GFP and mStrawberry were swapped using AgeI and EcoRI digestion. The plasmid was subsequently injected into the germline of lin-15(n765) animals to generate transgenic strains.

The ceh-22::mCherry and hlh-3::gfp translational reporters were constructed using fosmid recombineering as described (59). Briefly, mCherry or egfp coding sequence was amplified from the plasmid NM1845 pR6KmCherry or NM1835 pR6KGFP(59), respectively, using the oligonucleotides

fw-GACCTTCAGCAGCTTCTTCCTACATGACCAATACTCAATGGTGGCCTTCTGAATTCATGGTGAGCAAGGGC (SEQ ID NO: 3),
rv-GAGATGTATCTGGGAAAAATITGACATGGTATAGAGTATTAGAGAAATCAaccggcagatcgtcagtcag (ceh-22::mCherry; SEQ ID NO: 4), and
fw-CATCCACTTCTGGTGATCATCATAGCTTTTATTCGCATACAGAAACTTATagctcaggaggtagcggCA (SEQ ID NO: 5),
rv-CACCCGATTATTTGAGAAAAACAGAAAATATGGTACAACTTAACAGATTAaccggcagatcgtcagtcag (hlh-3::gfp; SEQ ID NO: 6).

The PCR products were digested with DpnI to remove template DNA, gel-purified by QIAquick gel extraction kit (Qiagen), and 1 μl of the purified products were electroporated into L-rhamnose-induced competent bacterial cells that harbored the helper plasmid pREDFlp4 and the fosmid containing full-length ceh-22 (fosmid 19b10) or hlh-3 (fosmid 40n18) genomic DNA. Successful recombinants with mCherry or egfp recombined into the fosmid were selected by kanamycin resistance, with the kan^{r} gene subsequently removed by anhydrotetracycline-induced Flp recombination. The correct insertion of mCherry or eGFP was verified by sequencing. The fosmids were subsequently injected into the germ line of wild-type animals to generate transgenic strains. The Pdpy-22::gfp transcriptional reporter was generated by overlap extension PCR that fused 2 kb dpy-22 promoter with the 0.7 kb egfp sequence from NM1847 pR6KKanRGFP, followed by 1 kb dpy-22 3′-UTR, using the oligonucleotides fw-ccacagcaaattcaaacatttcttg (SEQ ID NO: 7), rv-ATGGTGGCGACCGGTGCCATACGTTCGCCGGGCTGCTCGT (Pdpy-22; SEQ ID NO: 8), fw-ACGAGCAGCCCGGCGAACGTATGGCACCGGTCGCCACCAT (SEQ ID NO: 9), rv-GAAAGAATATAAATATGTAATTGTGACATGAttaTCCGCGGCCGTCCTIGT (egfp; SEQ ID NO: 10),

fw-ACAAGGACGGCCGCGGAtaaTCATGTCACAATTACATATTTATATTCTTC (SEQ ID NO: 11), and
rv-gcaggtggtacacataggaaag (dpy-22 3′-UTR SEQ ID NO: 12).

The PCR product was gel-purified using QIAquick gel extraction kit and subsequently injected into the germ line of wild-type animals to generate transgenic strains. The Plet-19::gfp transcriptional reporter was generated by overlap extension PCR that fused 1.8 kb let-19 promoter with the 0.7 kb egfp sequence from NM1847 pR6KKanRGFP, followed by 1.1 kb let-19 3′-UTR, using the oligonucleotides

fw-cgagaatgaacaaaaggtttctte (SEQ ID NO: 13),
rv-ATGGTGGCGACCGGTGCCATGTCCTCTGTGGAGTCACGGG (Plet-19; SEQ ID NO: 14),
fw-CCCGTGACTCCACAGAGGACATGGCACCGGTCGCCACCAT (SEQ ID NO: 15),
rv-GTACATTFGAAAATTFGATTCACGATATGCttaTCCGCGGCCGTCCTTGT (egfp; SEQ ID NO: 16),
fw-ACAAGGACGGCCGCGGAtaaGCATATCGTGAATCAAATTTTCAAATGTAC (SEQ ID NO: 17), and
rv-TGCAGATTCGGACGAAATTGGG (let-19 3′-UTR; SEQ ID NO: 18).

The PCR product was gel-purified using QIAquick gel extraction kit and subsequently injected into the germ line of wild-type animals to generate transgenic strains. The Pace-1::mCherry transcriptional reporter was generated by overlap extension PCR that fused 2 kb ace-1 promoter with the 0.9 kb mCherry sequence from pAA64, followed by 1.3 kb unc-54 3′-UTR from pPD122.56, using the oligonucleotides

fw-ggaagaagaagaagcagagaagaaa (SEQ ID NO: 19),
rv-CTTCTTCACCCTTTGAGACCATGCTTCTCAACATAATCGTITG (Pace-1; SEQ ID NO: 20),
fw-GATTATGATTTGTTGAAGAAGCATGGTCTCAAAGGGTGAAGAAG (SEQ ID NO: 21),
rv-CTCAGTTGGAATTcTACGAATGCTACTTATACAATTCATCCATGCC (mCherry; SEQ ID NO: 22),
fw-GGCATGGATGAATTGTATAAGTAGCATTCGTAgAATTCCAACTGAG (SEQ ID NO: 23), and
rv-GTCTCATGAGCGGATACATATTG (unc-54 3′-UTR; SEQ ID NO: 24).

The PCR product was gel-purified using QIAquick gel extraction kit and subsequently injected into the germ line of wild-type animals to generate transgenic strains. The Pdpy-22::cdk-8(cDNA, wt or KD)::dpy-22 3′-UTR rescue DNA was generated by overlap extension PCR that fused 2 kb dpy-22 promoter with the 1.8 kb cdk-8(wt or KD) cDNA sequence, followed by 2.2 kb dpy-22 3′-UTR, using the oligonucleotides

fw-ccacagcaaattcaaacatttcttg (SEQ ID NO: 25),
rv-TCATCAATCATTAATGTCATACGTTCGCCGGGCTGCTCGT (Pdpy-22; SEQ ID NO: 26),
fw-ACGAGCAGCCCGGCGAACGTATGACATTAATGATTGATGAAAACTTCA (SEQ ID NO: 27),
rv-ATAAATATGTAATGTGACATGATATCGATGATATTGTTGTGCCATTG (cdk-8, wt cDNA; SEQ ID NO: 28), or
fw-ACGAGCAGCCCGGCGAACGTATGACATTAATGATGATGAAAACTTCA (SEQ ID NO: 29),
rv-GATTCTTGAAAATCCCAAAGCAGCAATTTTTACCCT (SEQ ID NO: 30),
fw-AGGGTAAAAATTGCTGCTTTGGGATTTTCAAGAATC (SEQ ID NO: 31),
rv-ATAAATATGTAATTGTGACATGATTATCGATGATATTGTTGTTGCCATTG (cdk-8, D182A KD cDNA; SEQ ID NO: 32), and
fw-ACAACAATATCATCGATAATCATGTCACAATTACATATTTATATTCTTTC (SEQ ID NO: 33),
rv-gatgaggagtgccaaaggataaatg (dpy-22 3′-UTR; SEQ ID NO: 34).

The PCR products were gel-purified using QIAquick gel extraction kit and subsequently injected into the germ line of wild-type animals to generate transgenic strains.

The 2.4 kb his-9(SOD) genomic DNA fragment was generated by PCR-mediated mutagenesis using the oligonucleotides fw-cgctacagcaaacagcaatttaa (SEQ ID NO: 61), rv-TGGAGCCTTTCCTCCGGTGTCTTTACGGGCGGTTTGCTTA (Phis-9, SEQ ID NO: 62)), fw-TAAGCAAACCGCCCGTAAAGACACCGGAGGAAAGGCTCCA (SEQ ID NO: 63), and rv-caatgttttattctctgataaaaagtcaat (his-9(S10D), SEQ ID NO: 64)). The PCR product was gel-purified using a QIAquick gel extraction kit and the point mutation was verified by sequencing. It was subsequently injected into the germline of wild-type animals to generate transgenic strains.

The 3.7 kb his-71(S10D) genomic DNA fragment was generated by PCR-mediated mutagenesis using the oligonucleotides fw-gtgttgttccctttcattttagc (SEQ ID NO: 65), rv-AGGAGCTTTTCCTCCAGTGTCTTTACGCGCGGTTTGCTTG (Phis-71, SEQ ID NO: 66)), fw-CAAGCAAACCGCGCGTAAAGACACTGGAGGAAAAGCTCCT (SEQ ID NO: 67), and rv-cacacagaaatgcttccaacaaa

(his-71(S10D), SEQ ID NO: 68). The PCR product was gel-purified using a QIAquick gel extraction kit and the point mutation was verified by sequencing. It was subsequently injected into the germline of wild-type animals to generate transgenic strains.

The Pdpy-22::cyh-1(cDNA, AA)::dpy-22 3′-UTR rescue DNA was generated by overlap extension PCR that fused 2 kb dpy-22 promoter with the 1 kb cyh-1(AA) cDNA sequence, followed by 2.2 kb dpy-22 3′-UTR, using the oligonucleotides fw-ccacagcaaattcaaacatttcttg (SEQ ID NO: 7),

rv-TGTGTCGCCGTCGCGTACATACGTTCGCCGGGCTGCTCGT (Pdpy-22, SEQ ID NO: 69),
fw-ACGAGCAGCCCGGCGAACGTATGTACGCGACGGCGACACAAAAACG (SEQ ID NO: 70),
rv-GAATATAAATATGTAATTGTGACATGATCAATTAATTTCGTCATCCGCATCAACTGGC (cyh-IAA, SEQ ID NO: 71),
fw-GCGGATGACGAAATTAATTGATCATGTCACAATTACATATTTATATTCTrC (SEQ ID NO: 72), and
rv-gatgaggagtgccaaaggataaatg (dpy-22 3′-UTR, SEQ ID NO: 34)). The 5.2 kb final PCR product was gel-purified using a QIAquick gel extraction kit and the point mutations were verified by sequencing. It was subsequently injected into the germline of wild-type animals to generate transgenic strains.

The Pdpy-22::cyh-1(cDNA, DD)::dpy-22 3′-UTR rescue DNA was generated by overlap extension PCR that fused 2 kb dpy-22 promoter with the 1 kb cyh-1(DD) cDNA sequence, followed by 2.2 kb dpy-22 3′-UTR, using the oligonucleotides fw-ccacagcaaattcaaacatttcttg (SEQ ID NO: 7), rv-TGTGTGTCCGTCGCGTACATACGTrCGCCGGGCTGCTCGT (Pdpy-22, SEQ ID NO: 73), fw-ACGAGCAGCCCGGCGAACGTATGTACGCGACGGACACACAAAAACG (SEQ ID NO: 74),

rv-GAATATAAATATGTAATTGTGACATGATCAATTAATTrCGTCATCGTCATCAACTGGC (cyh-1DD, SEQ ID NO: 75),
fw-GACGATGACGAAATTAATTGATCATGTCACAATTACATATTTATATTCTTC (SEQ ID NO: 76), and
rv-gatgaggagtgccaaaggataaatg (dpy-22 3′-UTR, SEQ ID NO: 34). The 5.2 kb final PCR product was gel-purified using a QIAquick gel extraction kit and the point mutations were verified by sequencing. It was subsequently injected into the germline of wild-type animals to generate transgenic strains.

The Pdpy-22::cdk-7(cDNA, KD)::dpy-22 3′-UTR rescue DNA was generated by overlap extension PCR that fused 2

kb dpy-22 promoter with the 1.1 kb cdk-7(KD) cDNA sequence, followed by 2.2 kb dpy-22 3′-UTR, using the
oligonucleotides fw-ccacagcaaattcaaacatttcttg (SEQ ID NO: 7),
rv-GTATCGTAACGTCTACTCATACGTTCGCCGGGCTGCTCGT (Pdpy-22, SEQ ID NO: 77),
fw-ACGAGCAGCCCGGCGAACGTATGAGTAGACGTTACGATACAATA (SEQ ID NO: 78),
rv-CTCGATCCTAGTTTGATITTFGCAATAGCCACACATTCGCCCG (SEQ ID NO: 79),
fw-CGGGCGAATGTGTGGCTATTGCAAAAATCAAACTAGGATCGAGAGAA (SEQ ID NO: 80),
rv-ATAAATATGTAATTGTGACATGATTAATCAAAATTCAATCGTCGAACGG (cdk-7KD, SEQ ID NO: 81),
fw-GACGATFGAATITTGATTAATCATGTCACAATTACATATTATATTCTTfC (SEQ ID NO: 82), and
rv-gatgaggagtgccaaaggataaatg (dpy-22 3′-UTR, SEQ ID NO: 34). The 5.3 kb final PCR product was gel-purified using a QIAquick
gel extraction kit and the point mutation was verified by sequencing. It was subsequently injected into the germline
of wild-type animals to generate transgenic strains.

The Pdpy-22::cdk-7(cDNA, EE)::dpy-22 3′-UTR rescue DNA was generated by overlap extension PCR that fused 2 kb

dpy-22 promoter with the 1.1 kb cdk-7(EE) cDNA sequence, followed by 2.2 kb dpy-22 3′-UTR, using the
oligonucleotides fw-ccacagcaaattcaaacatttcttg (SEQ ID NO: 7),
rv-GTATCGTAACGTCTACTCATACGTTCGCCGGGCTGCTCGT (Pdpy-22, SEQ ID NO: 83),
fw-ACGAGCAGCCCGGCGAACGTATGAGTAGACGTTACGATACAATA (SEQ ID NO: 84),
rv-ACCTGATGCTCGTAAITrCTGITGGCTCTCCGAAGAATCGAGCCAAACC (SEQ ID NO: 85),
fw-TTCTTCGGAGAGCCAAACAGAAATTACGAGCATCAGGTTGTGACAAGATGGT (SEQ ID NO: 86),
rv-ATAAATATGTAATTGTGACATGATTAATCAAAATTCAATCGTCGAACGG (cdk-7EE, SEQ ID NO: 87),
fw-GACGATGAATTTGATTAATCATGTCACAATTACATATTATATTCTTTC (SEQ ID NO: 88), and
rv-gatgaggagtgccaaaggataaatg (dpy-22 3′-UTR, SEQ ID NO: 34). The 5.3 kb final PCR product was gel-purified using a QIAquick gel extraction kit and the point mutations were verified by sequencing. It was subsequently injected into the germline of wild-type animals to generate transgenic strains.

The bacterial strains expressing small interfering RNAs that target the following genes either were not available from the Ahringer(60) or the ORFeome(61) RNAi library or contained plasmids with incorrect inserts and were constructed as follows. The genomic DNA fragments spanning both exons and introns for these genes were amplified using the oligonucleotides fw-TCGCAAGCTTATGATGCCACGAATGGGACCT (SEQ ID NO: 35), rv-AGAGAAGCTTGACGTrGTTCTGGCAGTTGGT (mdt-6; SEQ ID NO: 36), fw-TCAGCAAGCTTCAAAGACGCTC (SEQ ID NO: 37), rv-AGAGAAGCTTCACATTCCGGAAAGCTCAATTC (mdt-9; SEQ ID NO: 38), fw-TCGCAAGCTTATGGATCCGAGTAGTCCGATG (SEQ ID NO: 39), rv-AGAGAAGCTTCGAGATCTTCTCTGATGCTTCT (mdt-10; SEQ ID NO: 40), fw-TCGCAAGCTTGTCCTCAACTTCAGCTGGAAAT (SEQ ID NO: 41), rv-AGAGAAGCTTGGAGTTICCAGTCCAAGATCTT (let-19; SEQ ID NO: 42), fw-GCAGAAGCTTTGGCTGCAGGAGCTCAATCAT (SEQ ID NO: 43), rv-AGAGAAGCTTCGAATCTTCAACGTCATTGCCA (rgr-1; SEQ ID NO: 44), fw-GCAGAAGCTTTCCCTAAATCAGCTGAACAGC (SEQ ID NO: 45), rv-AGAGAAGCTTTGTGCCCATTTCAACGAATCC (mdt-17; SEQ ID NO: 46), fw-TCGCAAGCTTATGATFCGAGTGGGCACAGCA (SEQ ID NO: 47), rv-AGAGAAGCTTGCGTAATTTTGTCGCGATCCG (mdt-20; (SEQ ID NO: 48), fw-TCGCAAGCTTACCTTCAACTGCAGGGAATCC (SEQ ID NO: 49), and rv-AGAGAAGCTTGAATCTCCATGTCAAATCACCC (mdt-27; SEQ ID NO: 50).

The PCR products were gel-purified using QIAquick gel extraction kit, digested with HindIII, ligated with HindIII digested RNAi vector pLA440(61), and transformed into HT115 E. coli cells. All RNAi clones were verified by sequencing.

### Mutagenesis Screen for I4 Mutants.

oxIs322; nIs310 LA larvae were mutagenized with ethyl methanesulfonate (EMS) as described previously(56). About 200,000 F2 or F3 animals were screened using a dissecting microscope equipped with UV light to detect GFP. The animals that lacked expression of GFP in the I4 cell, which is stereotypically located in the dorsal side of the posterior bulb of the pharynx in wild-type animals, were picked to single plates. The I4 GFP-negative phenotype of the mutants was verified in the next generation by analyzing both the GFP expression and the nuclear morphology of I4 using a Zeiss Axioskop2 compound microscope equipped with Nomarski differential interference contrast (DIC) optics. The complementation test and DNA sequence determination revealed that three mutants, n5469, n5564 and n5566 are alleles of hlh-3, five mutants, n5571, n5572, n5573, n5574 and n5662 are alleles of dpy-22, and two mutants, n5470 and n5563 are alleles of let-19.

### RNAi Treatments.

The RNAi experiments were performed by feeding the worms with bacteria expressing small interference RNAs as described previously(60, 61). Briefly, HT115 E. coli cells carrying RNAi clones were cultured overnight in LB liquid media supplemented with ampicillin. Thirty microliters of bacterial culture were seeded onto individual wells of the 24-well NGM plates supplemented with 1 mM IPTG and 75 mg/L ampicillin, and the plates were incubated at room temperature (22° C.) overnight (>12 hours) to induce siRNA expression. For the Mediator RNAi experiments, three to five L2 larvae were transferred to individual wells of the RNAi plates, grown at room temperature (22° C.) for three to four days, and the F1 progeny was scored for I4 GFP expression. The worms that lacked the GFP expression specifically in I4 were scored as I4-defective. The bacteria expressing the empty RNAi vector pL4440 was used as control.

### Microscopy.

Nomarski DIC and epifluorescence images were obtained using an Axioskop2 (Zeiss) compound microscope and OpenLab software (Agilent) and edited using Photoshop CS4 software (Adobe). For tracing embryonic lineages, 2- or 4-cell stage embryos were dissected from gravid hermaphrodites and mounted on a slide with a 5% agarose pad. The embryonic lineages were traced by direct observation of cell divisions and images were taken at appropriate time points. Confocal images were obtained using a Zeiss LSM 510 microscope and modified in ImageJ software (NIH) and Photoshop CS4 software (Adobe).

### Laser Microsurgery.

The laser ablation experiments were performed as described previously. Briefly, 2-cell stage embryos were dissected from gravid hermaphrodites and mounted on a slide with a 2% agarose pad. The embryos were allowed to divide to generate the P2 and E cells, and laser ablation of AB, P2 and E was performed as described (62). The embryos were then recovered, grown at 22° C. overnight and examined using a compound microscope for GFP reporter expression.

### Germline Transformation.

Transgenic lines were constructed using standard germline transformation procedures (63). All DNA samples were injected at a final concentration of 10 ng/μl. We used Punc-54::mCherry or Ppgp-12::4 xNLS::mCherry as a coinjection marker when needed at 5 ng/μl, and we co-injected pcDNA3 at 100 ng/μl for each injection.

### Western Blots.

Worms were grown on 100 mm plates with E. coli OP50 bacterial lawn until the E. coli was almost depleted; two plates of worms were harvested for each genotype. Worm pellets were flash-frozen in liquid nitrogen, thawed at room temperature, and resuspended in ice-cold 400 μl (final volume) of 1×SDS lysis buffer (2% SDS, 50 mM Tris pH6.8, 10% glycerol). The suspension was sonicated using a Fisher Scientific Sonicator (Model: FB120, 120 W, 20 k Hz) at 50% output for 3×5 second pulses with 1 minute intervals. Samples were then boiled at 95° C. for 20 minutes. 15 μg of proteins were resolved on a 4-15% Bio-Rad Mini-Protean TGX gel, transferred to a nitrocellulose membrane (Whatman Protran, 0.45 μm) and blotted with anti-phospho-H3S10 antibody (Millipore, 06-570) at 1:2000 dilution. The same membrane was stripped and re-blotted with anti-H3 antibody (Santa Cruz, sc-8654r) at 1:1000 dilution. Signals were developed using Chemiluminescence Reagent Plus Kit (PerkinElmer, NEL105), and images were captured with Bio-Rad ChemiDoc MP imaging system. All images were processed using Adobe Photoshop CS4.

### Yeast Two-Hybrid Assay.

The yeast two-hybrid assay was performed following the manufacturer's protocol (Clontech). Briefly, fresh Yeast Gold colonies (<1 week old) were cultured in YPD liquid medium at 30° C. to the O.D.600 of 0.5, harvested, washed, and resuspended in 1.1×TE/LiAc. 100 ng of bait and prey plasmids were mixed with 50 pg of denatured salmon sperm carrier DNA and were transformed into competent yeast cells in the presence of 1×PEG/LiAc. The cell mix was then plated on both -Leu-Trp and -Leu-Trp-His-Ade dropout plates and was allowed to grow and 30° C. for 2-3 days. Single colonies that grew on the double and quadruple dropout plates were resuspended in H2O and respotted to fresh dropout plates, which were grown at 30° C. for 2 days. Images of the respotted plates were captured using a Canon Powershot A590 digital camera (Canon) and processed by Photoshop CS4 software (Adobe).

### Results

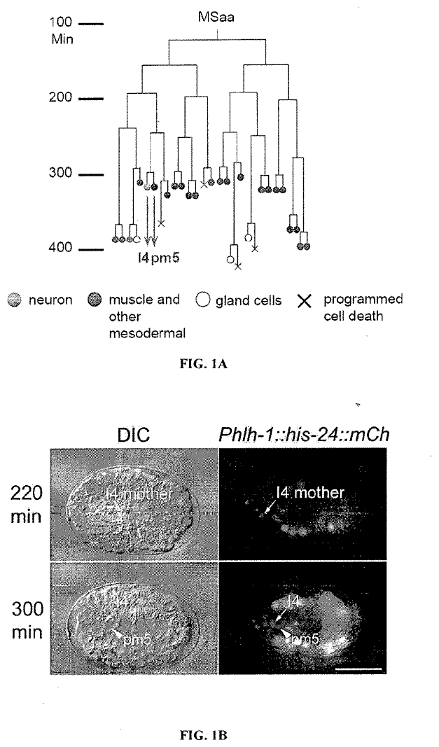

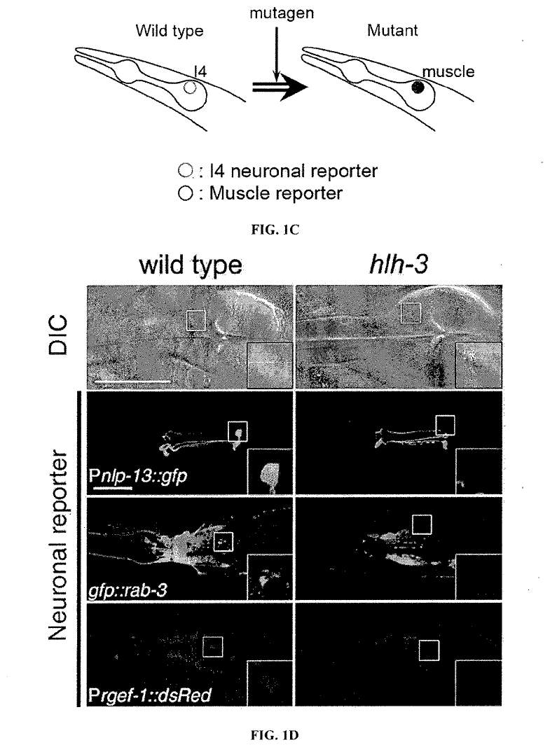

The nervous system of the C. elegans adult hermaphrodite consists of 302 neurons, 294 of which are derived from the AB founder cell of the early embryo (3). The AB cell gives rise to primarily hypodermal and neural cells and is considered to be ectodermal. Six C. elegans pharyngeal neurons are generated from the MS cell lineage, which primarily generate mesodermal cells, including muscle (FIG. 1A), and two are generated from the C lineage, which generates both ectoderm and mesoderm. We found that MS- and C-lineage neurons expressed reporters also expressed in AB-lineage ectodermal neurons—the small GTPase RAB-3 (gfp::rab-3) (4) and the guanine nucleotide exchange factor homolog RGEF-1 (Prgef-1::dsRed) (5)—suggesting that they are similar in basic neuronal identity (FIG. 1D and data not shown). One of the six pharyngeal neurons, the I4 neuron, is generated from a progenitor cell that divides to give rise to I4 and a pharyngeal muscle cell (FIG. 1A). We found that a transcriptional reporter for the C. elegans MyoD gene hlh-1 was expressed in I4 precursor cells during embryogenesis (FIG. 1B), and we hypothesized that the generation of I4 involves suppression of a mesodermal cell fate and/or promotion of a neuronal cell fate and chose to investigate the molecular mechanisms underlying I4 neuronal cell-fate specification.

We used reporter transgenes to label the I4 neuronal cell fate and the mesodermal cell fate of the I4 sister cell pm5. For I4 we generated a GFP reporter using the promoter of the neural peptide gene nlp-13 (6), and for pm5 we used a pharyngeal muscle myosin heavy-chain reporter Pmyo-2::mCherry, which labels pharyngeal muscle cells, including pm5 (7). We performed genetic screens for mutants that specifically lost I4 GFP expression and then identified those mutants with an extra pharyngeal muscle cell (FIG. 1C). Three such mutants carried alleles of the gene hlh-3, which encodes a bHLH transcription factor homologous to the mammalian proneural protein Ascl1/Mash1 (FIG. 1D). Ascl1 has been reported to be involved in neural development in flies and mammals, and overexpression of Ascl1 reportedly is associated with neuronal reprogramming from mammalian mesodermal and endodermal cells. The identification of hlh-3 as an important gene in I4 neuronal cell-fate specification establishes that the screen can identify factors involved in mammalian non-ectodermal neurogenesis (4, 8, 15-17).

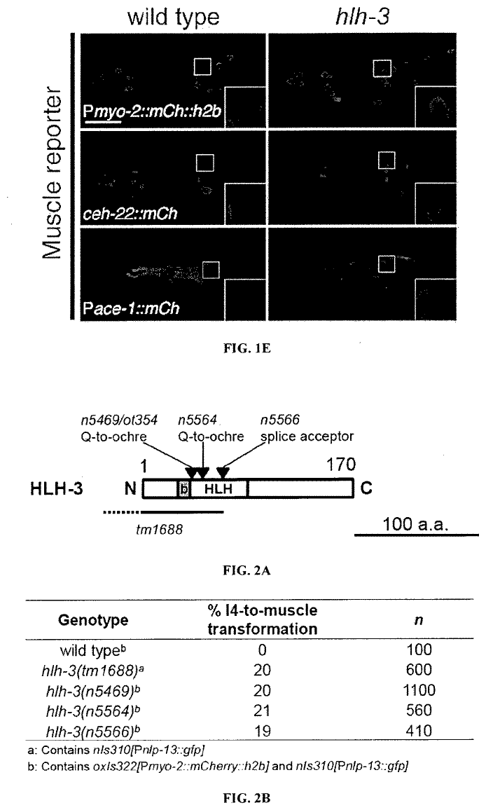

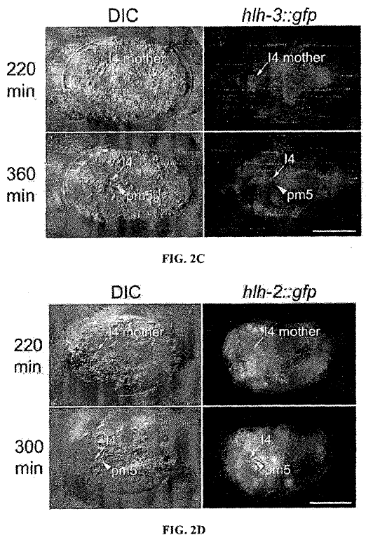

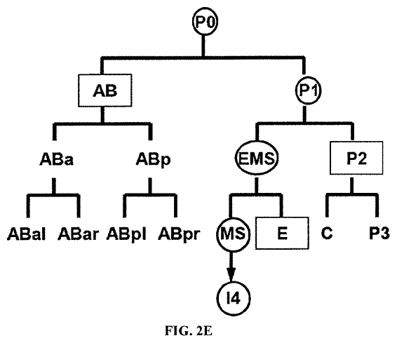

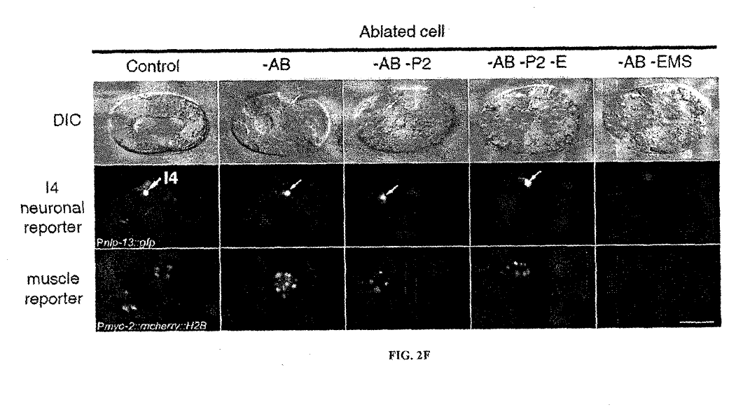

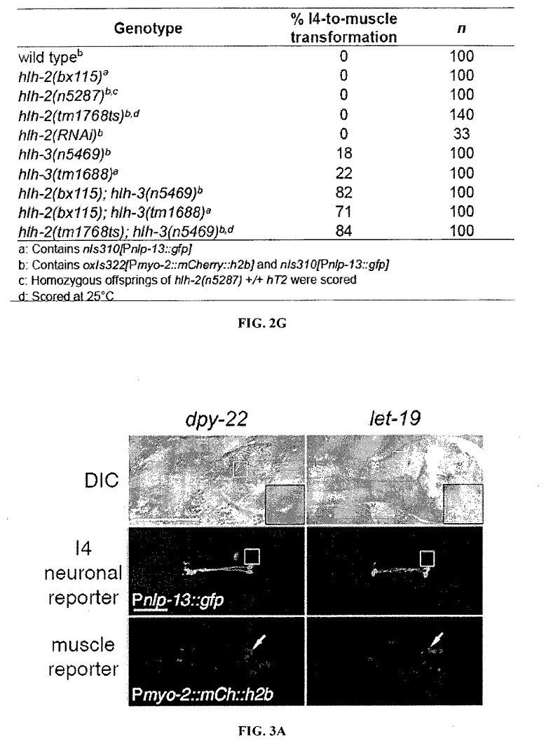

One hlh-3 allele, n5469, contains an early stop codon that truncates the protein before the evolutionarily conserved HLH domain and likely is a molecular null (FIG. 2A). The I4 cell in hlh-3 mutants appeared to have adopted a muscle-cell like fate: (1) the nuclear morphology of I4 as visualized using Nomarski optics was transformed from a neuronal speckled morphology to the fried-egg-like morphology characteristic of muscle and other non-neuronal cells (FIG. 1D); (2) the mutant I4 cell failed to express the three neuronal markers we examined, Pnlp-13::gfp, gfp::rab-3, and Prgef-1::dsRed (FIG. 1D); and (3) the mutant I4 cell expressed two pharyngeal muscle reporters, Pmyo-2::mCherry::His2B and ceh-22::mCherry (13, 14). (ceh-22 encodes a homologue of the mammalian Nkx2.5 transcription factor, which is involved in mammalian heart muscle development (15, 16); (FIG. 1E.) To further test whether I4 adopted the cell fate of its sister pharyngeal muscle cell pm5, we examined expression of the acetylcholine esterase reporter Pace-1::mCherry, which is expressed in pm5 (as well as in some other cells) (17). Whereas the wild-type pharynx contained six Pace-1::mCherry-expressing pm5 muscle cells, the hlh-3 mutant pharynx contained seven pm5 cells, and the extra pm5 appeared to fuse with the neighboring pm5 (just as pm5 cells normally fuse to form binuclear pharyngeal muscle cells in the wild type) (FIG. 1E). The cell-fate specification defect seems to be specific to I4, as we did not observe obvious defects for any of the 19 neuronal nuclei or for any of the 37 muscle nuclei in the hlh-3 pharynx (data not shown). Taken together, these results indicate that the I4 cell in hlh-3 mutants fails to be specified as a neuron and instead adopts the cell fate of its sister pm5 pharyngeal muscle cell.

Of the 20 neurons in the wild-type C. elegans pharynx, I4 are derived from ectoderm (from the AB lineage), and all I4 are generated normally in the three hlh-3 mutants (data not shown). To determine if HLH-3 regulates a proneural program in mesodermal lineages, we used available neurotransmitter reporter transgenes (for cholinergic, GABAergic, glutamatergic, dopaminergic, serotonergic and tyraminergic/octopaminergic neurons) and examined all six MS-derived neurons and about 220 AB-derived neurons. Of the six MS-derived neurons, only I4 was specifically missing from hlh-3 mutants. We found that about 10% of the wild-type animals variably expressed the glutamate transporter transgene eat-4::mCherry (but not any other neurotransmitter reporter transgenes) in the I4 neuron, indicating that I4 is probably glutamatergic. We did not find any major difference in the number of eat-4-expressing neurons between the wild type (78.1±1.0, mean±s.e.m, n=10) and hlh-2; hlh-3 (77.0±0.6, n=16) and hlh-3; dpy-22 (77.8±0.4, n=15) double mutant animals, indicating that the fates of most glutamatergic neurons were not altered. (We describe these hlh-3 double mutants below.) There similarly was no difference in cholinergic (wild type: 116.3±0.9, n=13; hlh-2; hlh-3: 115.5±1.0, n=15; hlh-3; dpy-22: 115.7±0.9, n=17), dopaminergic (wild type: 7.9±0.1, n=19; hlh-3: 8.0±0, n=20; hlh-2; hlh-3: 8.0±0, n=19), serotonergic (wild type: 4.0±0, n=20; hlh-3; dpy-22: 4.0±0.1, n=20; hlh-3: 4.2±0.1, n=20), or tyraminergic/octopaminergic neuron numbers (wild type: 4.0±0, n=20; hlh-2; hlh-3: 4.0±0.1, n=20; hlh-3; dpy-22: 4.0±0.1, n=20) between wild-type and hlh-3 mutant animals. We noticed a mild defect in GABAergic neuron specification in hlh-3 double mutants, which had 1 to 5 (mean: 1.3) fewer GABAergic ventral cord motor neurons than did wild-type animals (wild type: 18.8±0.1, n=25; hlh-2; hlh-3: 17.5±0.3, n=25; hlh-3; dpy-22: 17.5±0.2, n=25, P<0.001). In mammals, knockout of Ascl1 results in impaired neurogenesis in limited neural regions, including ventral telencephalon, olfactory bulb and autonomic ganglia, while neurogenesis in other brain regions remains grossly normal (12, 18). We conclude that like Ascl1, HLH-3 does not have general effects on neurogenesis. Rather, HLH-3 seems primarily to promote neuronal cell fate specification of I4 and a few GABAergic neurons.

We examined HLH-3 expression during embryogenesis. An HLH-3::GFP fusion protein was expressed in the I4 neuron shortly after its mother divided to generate I4; by contrast, the I4 sister, pm5, did not express HLH-3::GFP (FIG. 2C). We also observed expression of HLH-3::GFP in about 50 AB-derived neural precursors. To determine if HLH-3 functions within the I4 lineage or in neighboring cells to promote I4 neurogenesis, we used a laser microbeam to selectively kill the cells in physical contact with I4 progenitor cells during embryogenesis. We asked if elimination of any neighboring cells impairs I4 neurogenesis (FIG. 2E). Laser ablation of the founder cells AB, P2, and E, which normally generate neighbors of I4 progenitor cells in early embryos, did not affect I4 GFP reporter expression (FIG. 2F). As a control, killing the I4 progenitor cell EMS eliminated I4 GFP reporter expression (FIG. 2F). These results suggest that HLH-3, which is expressed specifically in I4, likely functions cell-autonomously to drive I4 neurogenesis.

The neurogenesis of I4 was only partially disrupted in the absence of functional HLH-3. Of the four hlh-3 mutants we examined, n5469 and tm1688 are likely molecular null (FIG. 2A). Nevertheless, in only about 20% of those mutant animals did I4 adopt a muscle cell fate (FIG. 2B). We reasoned that other genes must function in addition to hlh-3 to drive I4 neurogenesis. HLH-3 can interact and form heterodimers with another bHLH transcription factor HLH-2, which is the C. elegans homolog of the conserved E2A/Tcf3/Daughterless protein (19, 20). Tcf3 and Daughterless are broadly expressed in developing neural precursors in vertebrates and flies, respectively, and disruption of either protein results in loss of neural tissues and aberrant morphogenesis (26, 27, 28, 29). Consistent with previous findings (25), we observed that an HLH-2::GFP fusion protein was broadly expressed in neural precursor cells in early C. elegans embryos (FIG. 2D). Also, HLH-2::GFP was expressed in the I4 neuron shortly after its generation but was absent from its sister cell, pm5 (FIG. 2D). We asked if HLH-2 is required for I4 neurogenesis. The complete removal of HLH-2 function by genetic deletion (n5287) or partial reduction by RNAi resulted in embryonic lethality (data not shown); we did not observe obvious defects in I4 GFP expression in arrested hlh-2^{-/-} embryos (FIG. 2G). However, the introduction of an hlh-2 partial loss-of-function allele (bx115 or tm1768) into an hlh-3 null background significantly enhanced the penetrance of I4 misspecification, with about 80% of the I4 cells in hlh-2; hlh-3 double mutants adopting a muscle-like cell fate (FIG. 2G). We concluded that HLH-2 functions to promote I4 neurogenesis at least partly through a genetic pathway that acts in parallel to HLH-3.

The C. elegans genome encodes 42 bHLH factors; like HLH-3, the proneural proteins Neurogenin NGN-1 and NeuroD CND-1 can interact with HLH-2 (19). Disruption of mammalian Neurogenin and NeuroD leads to defects in neurogenesis and neuronal differentiation (26, 27). In C. elegans, NGN-1 promotes the specification of the fate of the AB-lineage neuron MI (vs. an epidermal cell fate) (25), while disruption of CND-1 results in absence of AB-derived ventral cord neurons (28). We did not observe defects in I4 neurogenesis in ngn-1, cnd-1 single mutants or in hlh-2; ngn-1 or hlh-2; cnd-1 double mutants. Given the different neurons affected by hlh-3, hlh-2, ngn-1 and end-1, we conclude that different proneural proteins promote the neurogenesis of different subsets of neurons in C. elegans.

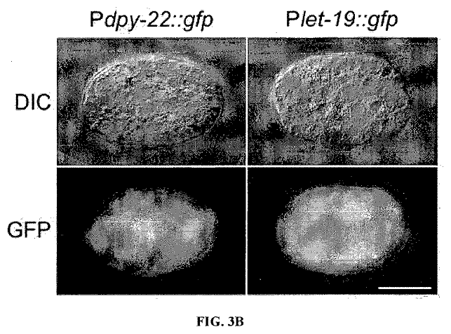

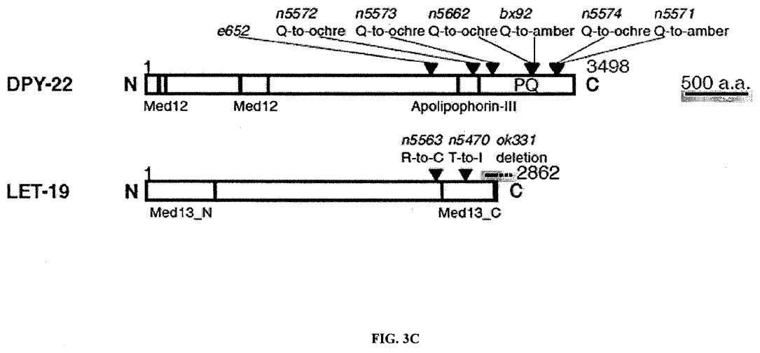

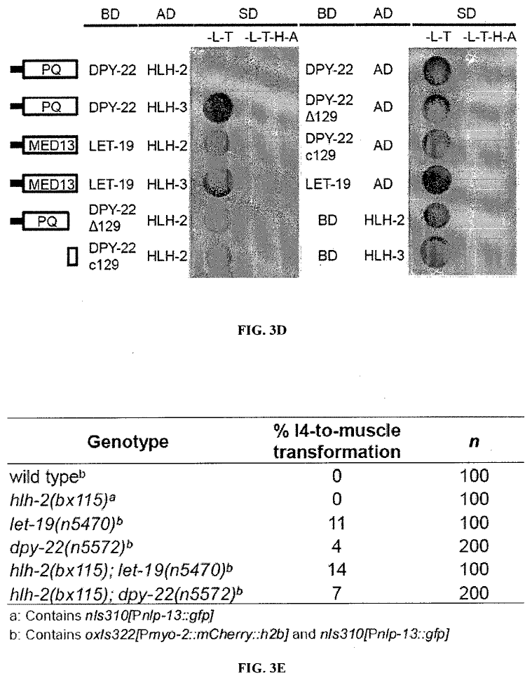

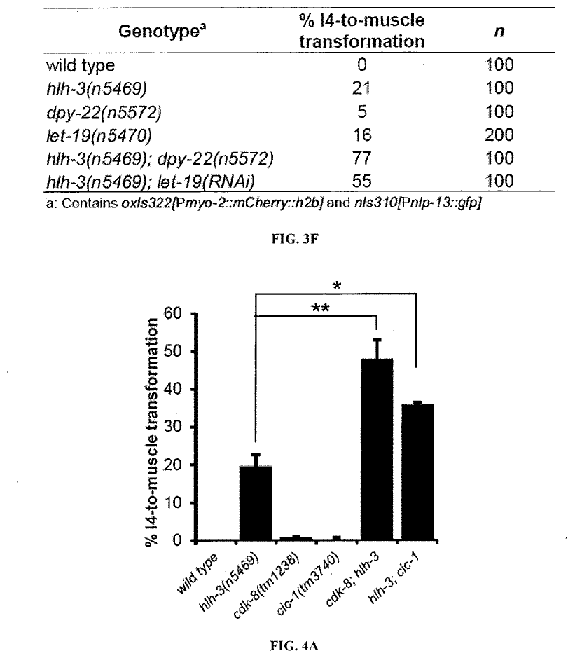

We examined other mutant isolates from the screens to seek additional factors that function with HLH-2 and HLH-3 to promote I4 neurogenesis. Five mutants carry alleles of dpy-22, and two carry alleles of let-19 (FIG. 3C). Like hlh-3 mutations, mutations in dpy-22 and let-19 specifically disrupted I4 specification, and the I4 cell adopted a pharyngeal muscle cell fate (FIG. 3A). dpy-22 and let-19 encode the worm homologs of the evolutionarily conserved Mediator subunits Med12 and Med13, respectively. Mediator is a multi-subunit complex that bridges DNA binding proteins (transcription factors/coactivators) with the RNA polymerase H transcription machinery and is involved in many aspects of gene regulation and animal development (29-31). Med12 disruption in mice and zebrafish results in impaired development of the neural crest and of non-ectodermal tissues, including heart and gut (32-36). Like HLH-3, DPY-22 has a specific role in promoting neurogenesis of I4 from mesoderm. Promoter-fusion reporter transgenes for dpy-22 and let-19 revealed broad GFP expression in developing embryos (FIG. 3B), suggesting that DPY-22 and LET-19 cooperate with cell-specific factors to drive I4 neurogenesis.

Two let-19 alleles contain missense mutations, and all five of the dpy-22 alleles contain nonsense mutations that truncate the C-terminal PQ-rich domain (FIG. 3C). In vertebrates, Med12 interacts with transcription factors through the PQ-rich domain to promote gene expression and tissue development (37-41). To determine if Mediator might specifically promote I4 neurogenesis by interacting with bHLH proneural factors, we performed a yeast two-hybrid assay. We found that the DPY-22 PQ-rich domain selectively interacted with HLH-2, but not HLH-3, while removal of the last 129 amino acids of the domain truncated in all five dpy-22 mutants eliminated the interaction (FIG. 3D). Further analysis indicated that the PQ-rich domain interacted with the N-terminal half of HLH-2, the region of a predicted transactivation domain important for gene expression and neurogenesis (42-45). These findings suggest that Mediator physically interacts with and functions in the same pathway as HLH-2 to promote I4 neurogenesis. To test this hypothesis, we constructed Mediator and bHLH double mutants. All the dpy-22 and let-19 single mutants had incompletely penetrant I4 misspecification, with only 5-16% of the I4 cells adopting a pharyngeal muscle cell fate. Introducing an hlh-2 mutation into dpy-22 or let-19 mutants did not enhance I4 misspecification (FIG. 3E). By contrast, disruption of dpy-22 or let-19 in an hlh-3 null (n5469) background significantly enhanced I4 misspecification, with 77% and 55% of the I4 cells adopting a muscle cell fate, respectively (let-19 and hlh-3 are tightly linked, and thus let-19 was tested using RNAi) (FIG. 3F). As a control, we performed dpy-22 or let-19 RNAi to further reduce gene function in dpy-22 or let-19 partial loss-of-function mutants, and we did not observe significant enhancement of the I4 misspecification. The results indicate that Mediator acts in the same pathway as HLH-2 to promote I4 neurogenesis and that HLH-2 likely recruits Mediator subunits through interactions with the PQ-rich domain of DPY-22.

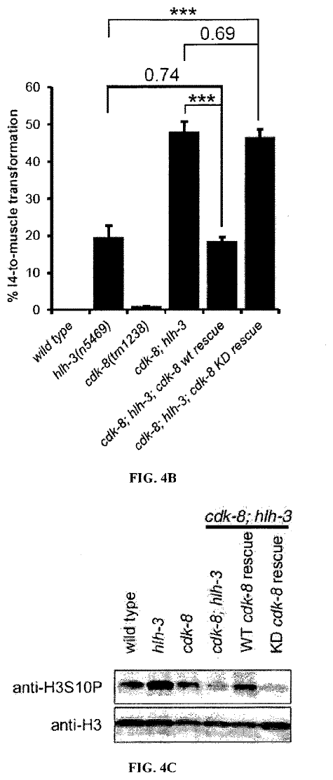

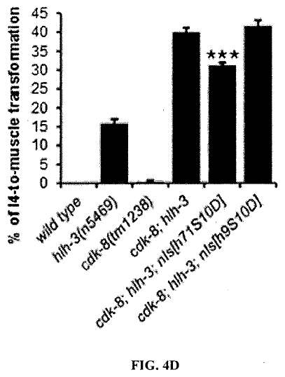

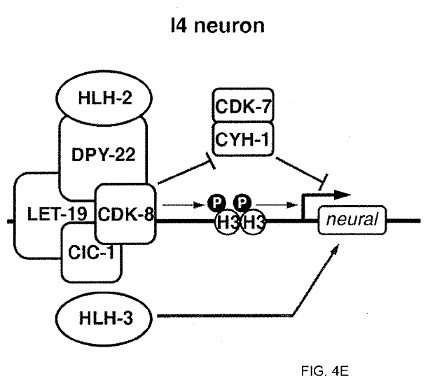

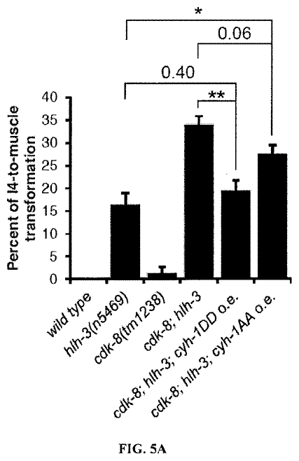

Med12 and Med13 are part of a four-protein Mediator submodule known as the kinase module (29, 30). The other two proteins, the cyclin-dependent kinase Cdk8 and cyclinC CycC, often co-purify with Med12 and Med13; CDK8 has also been found to be involved in tumor generation and progression (46, 47). To investigate if the nematode counterparts of Cdk8 and cyclinC are involved in I4 neurogenesis, we examined I4 development in cdk-8(tm1238) and cic-1(tm3740) mutants that contain deletions of coding exons and are likely nulls. cdk-8 and cic-1 single mutants had only very mild (<1%) defects in I4 neurogenesis. Introducing the cdk-8 or cic-1 allele into the Mediator or hlh-2 mutant did not enhance I4 misspecification. By contrast, disrupting cdk-8 or cic-1 in the hlh-3(n5469) null mutant significantly enhanced I4 misspecification, with 36% of the I4s in hlh-3; cic-1 mutants and 48% of the I4s in cdk-8; hlh-3 mutants adopting a muscle cell fate (FIG. 4A). We conclude that CDK-8 and CIC-1 function in the same pathway as DPY-22 and HLH-2 and in parallel to HLH-3 to promote I4 neurogenesis. We could fully rescue the enhanced I4 misspecification of cdk-8; hlh-3 double mutants with a wild-type, but not a kinase-dead, CDK-8 cDNA, suggesting that the kinase activity of CDK-8 is required for promoting I4 neurogenesis (FIG. 4B). As the penetrance of I4 misspecification in cdk-8, hlh-3 double mutants (˜40%) is only about half of that in hlh-3; dpy-22 (˜80%), we speculate that DPY-22 functions only partially through CDK-8 and CIC-1, with other unidentified proteins downstream of DPY-22 functioning in parallel to CDK-8 to promote I4 neurogenesis.

Previous studies showed that CDK8 can phosphorylate several substrates, including serine 10 of histone 3 (H3S10) (48, 49). Several lines of evidence indicate that phosphorylated H3S10 promotes dissociation of heterochromatin protein HP1 from heterochromatin and the opening of chromatin structure (50-53). We hypothesized that CDK-8 may promote I4 neurogenesis by maintaining open chromatin to facilitate neural gene expression. Consistent with this hypothesis, we observed that the phosphorylation level of H3S10 was significantly reduced in cdk-8; hlh-3 double mutants (FIG. 4C). We could restore H3S10 phosphorylation in these double mutants by expressing a wild-type, but not a kinase-dead, cdk-8 transgene (FIG. 4C). In addition, overexpression of a replication-independent His3.3 protein HIS-71 mutant form that mimics serine 10 phosphorylation (HIS-71S10D) partially suppressed I4 misspecification in cdk-8; hlh-3 double mutants, whereas overexpression of the phosphomimetic, replication-dependent His3.1 protein HIS-9 (HIS-9S10D) did not suppress the I4 defects (FIG. 4D). These findings support the hypothesis that CDK-8 promotes I4 neurogenesis at least partly through phosphorylation of serine 10 on replication-independent His3.3 (FIG. 4E).

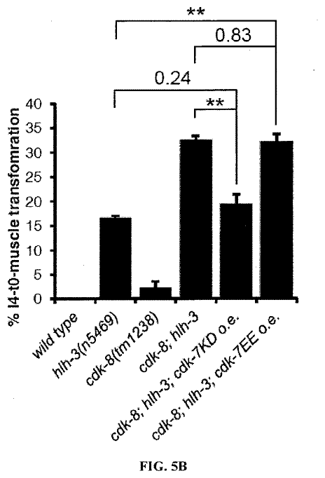

Mammalian CDK8 phosphorylates cyclin H on serines 5 and 304 and suppresses cyclin H/CDK7-activated gene transcription (Akoulitchev et al., 2000). Serine 5 (but not serine 304) of cyclin H is completely conserved from C. elegans to mammals. We asked if cyclin H might be a primary mediator of CDK-8 function. We generated a phosphomimetic (S5D S327D; “DD”) and a non-phosphorylatable (S5A S327A; “AA”) cyh-1 transgene (similar to S304 in mammals, S327 locates to the C-terminus of cyclin H) and found that overexpression of phosphomimetic CYH-1(DD) fully rescued the cdk-8; hlh-3 mutant phenotype, while overexpression of CYH-1(AA) did not rescue (FIG. 5A), indicating that CDK-8 might function primarily through cyclin H inhibition to promote I4 neurogenesis. As phosphorylation of cyclin H inhibits CDK7 kinase activity in the general transcription factor complex TFIIH (Akoulitchev et al., 2000), we tested if mutations that either enhance or reduce CDK7 kinase activity affect the cdk-8; hlh-3 mutant phenotype. We found that overexpression of a kinase-dead version of CDK-7, K34A (Garrett et al., 2001) resulted in complete rescue of the cdk-8; hlh-3 mutant phenotype, while overexpression of a constitutively active mutant of CDK-7 S157E T163E (“EE,” T-loop double mutations) (Garrett et al., 2001) did not have such an effect (FIG. 5B). Taken together, these results establish a strong link between CDK-8/CIC-1 and CDK-7/CYH-1. We conclude that CDK-8 functions to promote I4 neurogenesis primarily by inhibiting CDK-7/cyclin H and that H3S10 phosphorylation plays a secondary role.

The ability of non-ectodermal cells to generate neurons is a phenomenon with important implications for neuroregenerative medicine. In this study, we have analyzed the molecular genetic basis of neurogenesis from a mesodermal origin. We found that the proneural protein HLH-3, the mammalian homolog of which (Ascl1) can drive mammalian neuronal reprogramming, promotes I4 neurogenesis from mesoderm in C. elegans, establishing a similarity between I4 neurogenesis and mammalian neuronal reprogramming. We discovered that the Mediator CDK8 kinase submodule cooperates with HLH-3 to promote efficient non-ectodermal neurogenesis at least partly through CDK-8-mediated phosphorylation of serine 10 on His3.3. Given the high conservation of the proteins involved in C. elegans I4 neurogenesis with mammalian bHLH and Mediator proteins, an understanding of the molecular mechanisms underlying I4 neurogenesis will generate novel insights into neural development and may be used to identify novel factors useful in neuro-regenerative medicine.

### REFERENCES

1. Z. Wei, R. C. Angerer, L. M. Angerer, Direct development of neurons within foregut endoderm of sea urchin embryos. Proc. Natl. Acad. Sci. U.S.A. 108, 9143-7 (2011).
2. K. Seipel, V. Schmid, Evolution of striated muscle: Jellyfish and the origin of triploblasty. Dev. Biol. 282, 14-26 (2005).
3. J. E. Sulston, E. Schierenberg, J. G. White, J. N. Thomson, The embryonic cell lineage of the nematode Caenorhabditis elegans. Dev. Biol. 100, 64-119 (1983).
4. T. R. Mahoney et al., Regulation of synaptic transmission by RAB-3 and RAB-27 in Caenorhabditis elegans. Mol. Biol. Cell. 17, 2617-2625 (2006).
5. C. Bénard, N. Tjoe, T. Boulin, J. Recio, O. Hobert, The small, secreted immunoglobulin protein ZIG-3 maintains axon position in Caenorhabditis elegans. Genetics. 183, 917-27 (2009).
6. M. Rauthan, C. Mörck, M. Pilon, The C. elegans M3 neuron guides the growth cone of its sister cell M2 via the Krüppel-like zinc finger protein MNM-2. Dev. Biol. 311, 185-199 (2007).
7. C. Frøkjaer-Jensen et al., Single-copy insertion of transgenes in Caenorhabditis elegans. Nat. Genet. 40, 1375-83 (2008).
8. S. Marro et al., Direct lineage conversion of terminally differentiated hepatocytes to functional neurons. Cell Stem Cell. 9, 374-82 (2011).
9. T. Vierbuchen et al., Direct conversion of fibroblasts to functional neurons by defined factors. Nature. 463, 1035-41 (2010).
10. C. V. Cabrera, A. Martinez-Arias, M. Bate, The expression of three members of the achaete-scute gene complex correlates with neuroblast segregation in Drosophila. Cell. 50, 425-433 (1987).
11. F. Guillemot et al., Mammalian achaete-scute homolog 1 is required for the early development of olfactory and autonomic neurons. Cell. 75, 463-476 (1993).
12. S. Casarosa, C. Fode, F. Guillemot, Mash1 regulates neurogenesis in the ventral telencephalon. Development. 126, 525-534 (1999).
13. P. G. Okkema, E. Ha, C. Haun, W. Chen, A. Fire, The Caenorhabditis elegans NK-2 homeobox gene ceh-22 activates pharyngeal muscle gene expression in combination with pha-1 and is required for normal pharyngeal development. Development. 124, 3965-3973 (1997).
14. P. G. Okkema, S. W. Harrison, V. Plunger, A. Aryana, A. Fire, Sequence requirements for myosin gene expression and regulation in Caenorhabditis elegans. Genetics. 135, 385-404 (1993).
15. I. Lyons et al., Myogenic and morphogenetic defects in the heart tubes of murine embryos lacking the homeo box gene Nkx2-5. Genes Dev. 9, 1654-1666 (1995).
16. J. D. Mably, C. C. Liew, Factors involved in cardiogenesis and the regulation of cardiac specific gene expression. Circ. Res. 79, 4-13 (1996).
17. E. Culetto et al., Structure and promoter activity of the 5′ flanking region of ace-1, the gene encoding acetylcholinesterase of class A in Caenorhabditis elegans. J. Mol. Biol. 290, 951-66(1999).
18. F. Guillemot et al., Mammalian achaete-scute homolog 1 is required for the early development of olfactory and autonomic neurons. Cell. 75, 463-476 (1993).
19. C. a Grove et al., A multiparameter network reveals extensive divergence between C. elegans bHLH transcription factors. Cell. 138, 314-27 (2009).
20. M. Krause et al., A C. elegans E/Daughterless bHLH protein marks neuronal but not striated muscle development. Development. 124, 2179-89 (1997).
21. M. Caudy et al., The maternal sex determination gene daughterless has zygotic activity necessary for the formation of peripheral neurons in Drosophila. Genes Dev. 2, 843-852 (1988).
22. M. Caudy et al., daughterless, a Drosophila gene essential for both neurogenesis and sex determination, has sequence similarities to myc and the achaete-scute complex. Cell. 55, 1061-1067 (1988).
23. B. J. Merrill et al., Tcf3: a transcriptional regulator of axis induction in the early embryo. Development. 131, 263-274 (2004).
24. C. H. Kim et al., Repressor activity of Headless/Tcf3 is essential for vertebrate head formation. Nature. 407, 913-916 (2000).
25. S. Nakano, R. E. Ellis, H. R. Horvitz, Otx-dependent expression of proneural bHLH genes establishes a neuronal bilateral asymmetry in C. elegans. Development. 137, 4017-27 (2010).
26. M. H. Schwab et al., Neuronal basic helix-loop-helix proteins (NEX and BETA2/Neuro D) regulate terminal granule cell differentiation in the hippocampus. J. Neurosci. 20, 3714-3724 (2000).
27. Y. Sun et al., Neurogenin promotes neurogenesis and inhibits glial differentiation by independent mechanisms. Cell. 104, 365-376 (2001).
28. S. Hallam, E. Singer, D. Waring, Y. Jin, The C. elegans NeuroD homolog end-1 functions in multiple aspects of motor neuron fate specification. Development. 127, 4239-4252 (2000).
29. J. Yin, G. Wang, The Mediator complex: a master coordinator of transcription and cell lineage development. Development. 141, 977-87 (2014).
30. S. Malik, R. G. Roeder, The metazoan Mediator co-activator complex as an integrative hub for transcriptional regulation. Nat. Rev. Genet. 11, 761-72 (2010).
31. H. Bourbon et al., A unified nomenclature for protein subunits of mediator complexes linking transcriptional regulators to RNA polymerase II. Mol. Cell. 14, 553-7 (2004).
32. C. H. Shin et al., Multiple roles for Med12 in vertebrate endoderm development. Dev. Biol. 317, 467-479 (2008).
33. P. P. Rocha, M. Scholze, W. Bleiss, H. Schrewe, Med12 is essential for early mouse development and for canonical Wnt and Wnt/PCP signaling. Development. 137, 2723-2731 (2010).
34. M. J. Rau, S. Fischer, C. J. Neumann, Zebrafish Trap230/Med12 is required as a coactivator for Sox9-dependent neural crest, cartilage and ear development. Dev. Biol. 296, 83-93 (2006).
35. S.-K. Hong et al., The zebrafish kohtalo/trap230 gene is required for the development of the brain, neural crest, and pronephric kidney. Proc. Natl. Acad. Sci. U.S.A. 102, 18473-18478 (2005).
36. X. Wang, N. Yang, E. Uno, R. G. Roeder, S. Guo, A subunit of the mediator complex regulates vertebrate neuronal development. Proc. Natl. Acad. Sci. U.S.A. 103, 17284-17289 (2006).
37. N. Ding et al., Mediator links epigenetic silencing of neuronal gene expression with x-linked mental retardation. Mol. Cell. 31, 347-59 (2008).
38. S. Kim, X. Xu, A. Hecht, T. G. Boyer, Mediator is a transducer of Wnt/beta-catenin signaling. J. Biol. Chem. 281, 14066-75 (2006).
39. H. Zhou, S. Kim, S. Ishii, T. G. Boyer, Mediator modulates Gli3-dependent Sonic hedgehog signaling. Mol. Cell. Biol. 26, 8667-82 (2006).
40. H. Zhang, S. W. Emmons, A C elegans mediator protein confers regulatory selectivity on lineage-specific expression of a transcription factor gene. Genes Dev. 14, 2161-72 (2000).
41. R. Zhou et al., SOX9 interacts with a component of the human thyroid hormone receptor-associated protein complex. Nucl. Acids Res. 30, 3245-52 (2002).
42. J. W. Cave, L. Xia, M. a Caudy, The Daughterless N-terminus directly mediates synergistic interactions with Notch transcription complexes via the SPS+A DNA transcription code. BMC Res. Notes. 2, 65 (2009).
43. I. Zarifi et al., Essential roles of Da transactivation domains in neurogenesis and in E(spl)-mediated repression. Mol. Cell. Biol. 32, 4534-48 (2012).
44. M. E. Massari, P. A. Jennings, C. Murre, The AD1 transactivation domain of E2A contains a highly conserved helix which is required for its activity in both Saccharomyces cerevisiae and mammalian cells. Mol. Cell. Biol. 16, 121-9 (1996).
45. M. W. Quong, M. E. Massari, R. Zwart, C. Murre, A new transcriptional-activation motif restricted to a class of helix-loop-helix proteins is functionally conserved in both yeast and mammalian cells. Mol. Cell. Biol. 13, 792-800 (1993).
46. A. Kapoor et al., The histone variant macroH2A suppresses melanoma progression through regulation of CDK8. Nature. 468, 1105-1109 (2010).
47. R. Firestein et al., CDK8 is a colorectal cancer oncogene that regulates beta-catenin activity. Nature. 455, 547-551 (2008).
48. M. T. Knuesel, K. D. Meyer, A. J. Donner, J. M. Espinosa, D. J. Taatjes, The human CDK8 subcomplex is a histone kinase that requires Med12 for activity and can function independently of mediator. Mol. Cell. Biol. 29, 650-661 (2009).
49. K. D. Meyer et al., Cooperative activity of cdk8 and GCN5L within Mediator directs tandem phosphoacetylation of histone H3. EMBO J. 27, 1447-1457 (2008).
50. W. Fischle et al., Regulation of HP1-chromatin binding by histone H3 methylation and phosphorylation. Nature. 438, 1116-1122 (2005).
51. H. Deng et al., Ectopic histone H3S10 phosphorylation causes chromatin structure remodeling in Drosophila. Development. 135, 699-705 (2008).
52. W. Zhang et al., The JIL-1 histone H3S10 kinase regulates dimethyl H3K9 modifications and heterochromatic spreading in Drosophila. Development. 133, 229-235 (2006).
53. T. Hirota, J. J. Lipp, B.-H. Toh, J.-M. Peters, Histone H3 serine 10 phosphorylation by Aurora B causes HP1 dissociation from heterochromatin. Nature. 438, 1176-1180 (2005).
54. R. Doonan, J. Hatzold, S. Raut, B. Conradt, A. Alfonso, HLH-3 is a C. elegans Achaete/Scute protein required for differentiation of the hermaphrodite-specific motor neurons. Mech. Dev. 125, 883-893 (2008).
55. M. Thellmann, J. Hatzold, B. Conradt, The Snail-like CES-1 protein of C. elegans can block the expression of the BH3-only cell-death activator gene egl-1 by antagonizing the function of bH LH proteins. Development. 130, 4057-4071 (2003).
56. S. Brenner, The genetics of Caenorhabditis elegans. Genetics. 77, 71-94 (1974).
57. E. Serrano-Saiz et al., Modular control of glutamatergic neuronal identity in C. elegans by distinct homeodomain proteins. Cell. 155, 659-673 (2013).
58. D. L. Chase, J. S. Pepper, M. R. Koelle, Mechanism of extrasynaptic dopamine signaling in Caenorhabditis elegans. Nat. Neurosci. 7, 1096-1103 (2004).
59. M. Sarov et al., A recombineering pipeline for functional genomics applied to Caenorhabditis elegans. Nat. Methods. 3, 839-44 (2006).
60. R. S. Kamath et al., Systematic functional analysis of the Caenorhabditis elegans genome using RNAi. Nature. 421, 231-7 (2003).
61. J. Rual et al., Toward improving Caenorhabditis elegans phenome mapping with an ORFeome-based RNAi library. Genome Res. 14, 2162-8 (2004).
62. L. Avery, H. R. Horvitz, A cell that dies during wild-type C. elegans development can function as a neuron in a ced-3 mutant. Cell. 51, 1071-8 (1987).
63. C. Mello, J. Kramer, D. Stinchcomb, V. Ambros, Efficient gene transfer in C. elegans: extrachromosomal maintenance and integration of transforming sequences. EMBO J. 10, 3959-3970 (1991).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for generating neuronal cells from non-neuronal cells comprising
enhancing expression of exogenous ASCL1/HLH3 protein and CDK8 protein in non-neuronal cells at a level and for a period of time sufficient for the appearance of neuronal cells.

2. The method of claim 1, further comprising enhancing expression of exogenous TCF3/HLH2 protein in the non-neuronal cells.

3. A method for generating neuronal cells from non-neuronal cells comprising
enhancing expression of exogenous ASCL1/HLH3 protein and reducing activity of a CDK7/CYH1 complex in non-neuronal cells at a level and for a period of time sufficient for the appearance of neuronal cells.

4. The method of claim 3, further comprising enhancing expression of exogenous TCF3/HLH2 protein in the non-neuronal cells.

5. The method of claim 3 or 4, further comprising enhancing expression of exogenous CDK8 protein in the non-neuronal cells.

6. The method of claim 3, 4 or 5, wherein reducing activity of a CDK7/CYH1 complex comprises introducing a CDK7 and/or a cyclin H inhibitor into the non-neuronal cells.

7. A method for generating neuronal cells from non-neuronal cells comprising
enhancing expression of exogenous ASCL1/HLH3 protein and CDK8 protein and reducing activity of a CDK7/CYH1 complex in non-neuronal cells at a level and for a period of time sufficient for the appearance of neuronal cells.

8. The method of claim 7, further comprising enhancing expression of exogenous TCF3/HLH2 protein in the non-neuronal cells.

9. The method of claim 7 or 8, wherein reducing activity of a CDK7/CYH1 complex comprises introducing a CDK7 and/or a cyclin H inhibitor into the non-neuronal cells.

10. A method for generating neuronal cells from non-neuronal cells comprising
enhancing expression of exogenous ASCL1/HLH3 protein, TCF3/HLH2 protein, and CDK8 protein in non-neuronal cells at a level and for a period of time sufficient for the appearance of neuronal cells.

11. A method for generating neuronal cells from non-neuronal cells comprising
enhancing expression of exogenous ASCL1/HLH3 protein and TCF3/HLH2 protein and reducing activity of a CDK7/CYH1 complex in non-neuronal cells at a level and for a period of time sufficient for the appearance of neuronal cells.

12. The method of claim 11, wherein reducing activity of a CDK7/CYH1 complex comprises introducing a CDK7 and/or a cyclin H inhibitor into the non-neuronal cells.

13. A method for generating neuronal cells from non-neuronal cells comprising
enhancing expression of exogenous ASCL1/HLH3 protein, TCF3/HLH2 protein and CDK8 protein and reducing activity of a CDKY7/CYH1 complex in non-neuronal cells at a level and for a period of time sufficient for the appearance of neuronal cells.

14. The method of claim 13, wherein reducing activity of a CDK7/CYH1 complex comprises introducing a CDK7 and/or a cyclin H inhibitor into the non-neuronal cells.

15. The method of any one of claims 1-14, further comprising enhancing expression of exogenous MED12/DPY22 protein and/or MED13/LET19 protein in the non-neuronal cells.

16. The method of any one of claims 1-15, further comprising enhancing expression of exogenous CYCC/CIC1 protein in the non-neuronal cells.

17. A method for generating neuronal cells from non-neuronal cells comprising
enhancing expression of exogenous ASCL1/HLH3 protein and CYCC/CIC1 protein in non-neuronal cells at a level and a period of time sufficient for the appearance of neuronal cells.

18. The method of claim 17, further comprising enhancing expression of exogenous TCF3/HLH2 protein in the non-neuronal cells.

19. A method for generating neuronal cells from non-neuronal cells comprising
enhancing expression of exogenous ASCL1/HLH3 protein, TCF3/HLH2 protein, and CYCC/CIC1 protein in non-neuronal cells at a level and a period of time sufficient for the appearance of neuronal cells.

20. The method of any one of claims 17-19, further comprising enhancing expression of exogenous MED12/DPY22 protein and/or MED13/LET19 protein in the non-neuronal cells.

21. The method of any one of claims 17-20, further comprising enhancing expression of exogenous CDK8 protein in the non-neuronal cells.

22. A method for generating neuronal cells from non-neuronal cells comprising
enhancing expression of a ASCL1/HLH3-CDK8 fusion protein in non-neuronal cells at a level and a period of time sufficient for the appearance of neuronal cells.

23. The method of claim 22, wherein the fusion protein comprises full length ASCL1/HLH3 protein.

24. The method of claim 22 or 23, further comprising enhancing expression of exogenous TCF3/HLH2 protein in the non-neuronal cells.

25. The method of any one of claims 22-24, further comprising enhancing expression of exogenous MED12/DPY22 protein and/or MED13/LET19 protein in the non-neuronal cells.

26. The method of any one of claims 22-25, further comprising enhancing expression of exogenous CYCC/CIC1 protein in the non-neuronal cells.

27. A method for generating neuronal cells from non-neuronal cells comprising
enhancing expression of a ASCL1/HLH3-CYCC/CIC1 fusion protein in non-neuronal cells at a level and a period of time sufficient for the appearance of neuronal cells.

28. The method of claim 27, wherein the fusion protein comprises full length ASCL1/HLH3 protein.

29. The method of claim 27 or 28, further comprising enhancing expression of exogenous TCF3/HLH2 protein in the non-neuronal cells.

30. The method of any one of claims 27-29, further comprising enhancing expression of exogenous MED12/DPY22 protein and/or MED13/LET19 protein in the non-neuronal cells.

31. The method of any one of claims 27-30, further comprising enhancing expression of exogenous CDK8 protein in the non-neuronal cells.

32. A method for generating neuronal cells from non-neuronal cells comprising
enhancing expression of exogenous ASCL1/HLH3 protein and TCF3/HLH2 protein in non-neuronal cells at a level and a period of time sufficient for the appearance of neuronal cells.

33. The method of claim 32, further comprising enhancing expression of exogenous MED12/DPY22 protein and/or MED13/LET19 protein in the non-neuronal cells.

34. The method of claim 32 or 33 further comprising enhancing expression of exogenous CDK8 protein in the non-neuronal cells.

35. The method of any one of claims 32-34, further comprising enhancing expression of exogenous CYCC/CIC protein in the non-neuronal cells.

36. The method of any one of the preceding claims, wherein the non-neuronal cells are fibroblasts.

37. The method of any one of the preceding claims, wherein the non-neuronal cells are hepatocytes.

38. The method of any one of the preceding claims, wherein the exogenous proteins or fusion proteins are expressed using a viral expression construct.

39. The method of claim 38, wherein the viral expression construct is an adenoviral expression construct.

40. The method of claim 38, wherein the viral expression construct is a CM V expression construct.

41. The method of any one of the preceding claims, wherein the exogenous proteins are expressed from the same expression construct.

42. The method of any one of the preceding claims, wherein the exogenous proteins are expressed from separate expression constructs.

43. The method of any one of the preceding claims, further comprising enhancing expression of one or more Mediator subunit proteins.

44. The method of claim 43, wherein the Mediator subunit protein is selected from the group consisting of MED1, MED4, MED6, MED7, MED8, MED9, MED10, MED11, MED12, MED13, MED13L, MED14, MED15, MED16, MED17, MED18, MED19, MED20, MED21, MED22, MED23, MED24, MED25, MED26, MED27, MED28, MED29, MED30, MED31, CCNC and CDK8.

45. The method of any one of the preceding claims, further comprising enhancing expression of one or more Mediator CDK8 kinase module subunit proteins.

46. The method of claim 45, further comprising enhancing expression of all Mediator CDK8 kinase module subunit proteins.

47. The method of any one of the preceding claims, wherein the neuronal cells are produced with an efficiency of at least 25%.

48. The method of any one of the preceding claims, further comprising differentiating the neuronal cells in vitro.

49. The method of claim 48, further comprising analyzing the developmental potential of the neuronal cells.

50. The method of any one of claims 17-49, further comprising reducing activity of a CDK7/CYH1 complex in the non-neuronal cells.

51. The method of claim 50, wherein reducing activity of a CDK7/CYH1 complex comprises introducing a CDK7 and/or a cyclin H inhibitor into the non-neuronal cells

52. A method of diagnosing a subject at risk of developing a neurodegenerative disease comprising
reprogramming a non-neuronal cell from a subject into a neuronal cell by enhancing expression of exogenous
(i) ASCL1/HLH3 protein and CDK8 protein;
(ii) ASCL1/HLH3 protein, TCF3/HLH2 protein, and CDK8 protein;
(iii) ASCL1/HLH3 protein and CYCC/CIC1 protein;
(iv) ASCL1/HLH3 protein, TCF3/HLH2 protein, and CYCC/CIC1 protein;
(v) ASCL1/HLH3-CDK8 fusion protein; or
(vi) ASCL/HLH3-CYCC/CIC1 fusion protein,
differentiating the neuronal cell in vitro, and
analyzing the differentiated neuronal cell for the presence of markers associated with a neurodegenerative disease.

53. A method of diagnosing a subject at risk of developing a neurodegenerative disease comprising
reprogramming a non-neuronal cell from a subject into a neuronal cell by enhancing expression of exogenous
(i) ASCL1/HLH3 protein and CDK8 protein;
(ii) ASCL1/HLH3 protein, TCF3/HLH2 protein, and CDK8 protein;
(iii) ASCL1/HLH3 protein and CYCC/CIC1 protein;
(iv) ASCL1/HLH3 protein, TCF3/HLH2 protein, and CYCC/CIC1 protein;
(v) ASCL1/HLH3-CDK8 fusion protein; or
(vi) ASCL/HLH3-CYCC/CIC1 fusion protein,
reducing the activity of CDK7/CYH1 protein,
differentiating the neuronal cell in vitro, and
analyzing the differentiated neuronal cell for the presence of markers associated with a neurodegenerative disease.

54. The method of claim 52 or 53, wherein the neurodegenerative disease is selected from the group consisting of amyotrophic lateral sclerosis, Parkinson's disease, Alzheimer's disease, and Huntington's disease.

55. The method of claim 52, 53 or 54, wherein the subject is mammalian.

56. The method of claim 52, 53 or 54, wherein the subject is human.

57. A method for generating neuronal cells from non-neuronal cells comprising increasing activity of CDK8 mediator kinase module to a level and for a period of time sufficient for the appearance of neuronal cells.

58. The method of claim 57, wherein increasing activity of CDK8 mediator kinase module comprises increasing expression of one or more endogenous or exogenous CDK8 protein, CIC1 protein, MED12 protein, and MED13 protein.

59. A method for generating neuronal cells from non-neuronal cells comprising increasing activity of CDK8 protein to a level and for a period of time sufficient for the appearance of neuronal cells.

60. The method of claim 59, wherein increasing activity of CDK8 protein comprises increasing expression of endogenous or exogenous CDK8 protein.

61. The method of any one of claims 57-60, further comprising increasing activity of TCF3/HLH2 protein in the non-neuronal cells.

62. The method of claim 61, wherein increasing activity of TCF3/HLH2 protein comprises increasing expression of endogenous or exogenous TCF3/HLH2 protein.

63. The method of any one of claims 57-62, further comprising decreasing activity of CDK7/CYH1 complex in the non-neuronal cells.

64. The method of claim 63, wherein decreasing activity of CDK7/CYH1 complex comprises decreasing expression of endogenous or exogenous CDK7 protein and/or CYH1 protein.

65. The method of claim 63, wherein decreasing activity of CDK7/CYH1 complex comprises introducing a CDK7 or CYH1 inhibitor into the non-neuronal cells.

66. A method for generating neuronal cells from non-neuronal cells comprising increasing activity of TCF3/HLH2 to a level and for a period of time sufficient for the appearance of neuronal cells.

67. The method of claim 66, wherein increasing activity of TCF3/HLH2 protein comprises increasing expression of endogenous or exogenous TCF3/HLH2 protein.

68. The method of claim 66 or 67, further comprising increasing activity of CDK8 mediator kinase module, optionally wherein this comprises increasing expression of one or more endogenous or exogenous CDK8 protein, CIC1 protein, MED12 protein, and MED13 protein.

69. The method of any one of claims 66-68, further comprising increasing activity of CDK8 protein, optionally wherein increasing activity of CDK8 protein comprises increasing expression of endogenous or exogenous CDK8 protein.

70. The method of any one of claims 66-69, further comprising decreasing activity of CDK7/CYH1 complex in the non-neuronal cells.

71. The method of claim 70, wherein decreasing activity of CDK7/CYH1 complex comprises decreasing expression of endogenous or exogenous CDK7 protein and/or CYH1 protein.

72. The method of claim 71, wherein decreasing activity of CDK7/CYH1 complex comprises introducing a CDK7 or CYH1 inhibitor into the non-neuronal cells.

73. A method for generating neuronal cells from non-neuronal cells comprising
decreasing activity of CDK7/CYH1 complex to a level and for a period of time sufficient for the appearance of neuronal cells.

74. The method of claim 73, wherein decreasing activity of CDK7/CYH1 complex comprises decreasing expression of endogenous or exogenous CDK7 protein and/or CYH1 protein.

75. The method of claim 73, wherein decreasing activity of CDK7/CYH1 complex comprises introducing a CDK7 or CYH1 inhibitor into the non-neuronal cells.

76. The method of any one of claims 73-75, further comprising increasing activity of TCF3/HLH2 protein in the non-neuronal cells.

77. The method of claim 76, wherein increasing activity of TCF3/HLH2 protein comprises increasing expression of endogenous or exogenous TCF3/HLH2 protein.

78. The method of any one of claims 73-77, further comprising increasing activity of CDK8 mediator kinase module, optionally wherein this comprises increasing expression of one or more endogenous or exogenous CDK8 protein, CIC1 protein, MED12 protein, and MED13 protein.

79. The method of any one of claims 73-78, further comprising increasing activity of CDK8 protein, optionally wherein increasing activity of CDK8 protein comprises increasing expression of endogenous or exogenous CDK8 protein.

80. A method for reducing neurogenesis comprising
decreasing activity of CDK8 mediator kinase module, or decreasing activity of TCF3/HLH2, or decreasing activity of CDK8 kinase, or increasing activity of CDK7/CYH1 complex in neuronal cells or cells fated to become neuronal cells, to a level and for a time sufficient for the appearance of non-neuronal cells.

81. The method of claim 80, comprising
(i) decreasing activity of CDK8 mediator kinase module and decreasing activity of TCF3/HLH2, or
(ii) decreasing activity of CDK8 mediator kinase module and increasing activity of CDK7/CYH1 complex, or
(iii) decreasing activity of TCF3/HLH2 and increasing activity of CDK7/CYH1 complex.
